(19) United States
(12) Patent Application Publication
(10) Pub. No.: US 2012/0141461 A1
(43) Pub. Date: Jun. 7, 2012

(54) COMPOSITIONS AND METHODS FOR DIAGNOSING AND TREATING FIBROTIC DISORDERS

(75) Inventors: Victor J. Thannickal, Birmingham, AL (US); Louise Hecker, Birmingham, AL (US)

(73) Assignee: THE REGENTS OF THE UNIVERSITY OF MICHIGAN, Ann Arbor, MI (US)

(21) Appl. No.: 13/384,108

(22) PCT Filed: Jul. 19, 2010
(86) PCT No.: PCT/US10/42432

§ 371 Date: Feb. 12, 2012

Related U.S. Application Data
(60) Provisional application No. 61/226,508, filed on Jul. 17, 2009.

(51) Int. Cl. A61K 31/713; A61P 9/00; A61K 39/395

(52) U.S. Cl. 424/130.1; 514/44 A

(57) ABSTRACT

The present invention relates to biomarkers, therapeutic targets, and therapeutic agents for treating and diagnosing firotic disorders. In particular, the present invention relates to diagnosis, drug screening, and therapeutic targeting of NOX4 biomarkers of pulmonary fibrosis and other fibrotic diseases and conditions.

### CROSS REFERENCE TO RELATED APPLICATIONS

The present application is a national phase application under 35 U.S.C. §371 of PCT International Application Number PCT/US2010/042432, filed on Jul. 19, 2010, which claims priority to U.S. Provisional Patent Application Ser. No. 61/226,508 filed Jul. 17, 2009, each of which are herein incorporated by reference in its entirety.

### GOVERNMENT SUPPORT

This invention was made with government support under HL067967 awarded by the National Institutes of Health. The Government has certain rights in the invention.

### FIELD OF THE INVENTION

The present invention relates to biomarkers, therapeutic targets, and therapeutic agents for treating and diagnosing fibrotic disorders. In particular, the present invention relates to diagnosis, drug screening, and therapeutic targeting of NOX4 biomarkers of pulmonary fibrosis and other fibrotic diseases and conditions.

### BACKGROUND OF THE INVENTION

Tissue repair in mammals involves the integrated actions of growth factors and matrix molecules that orchestrate cell-cell interactions. Fibrogenesis, the development or proliferation of fibers or fibrous tissue, occurs as a normal cellular process to generate fibrous tissue as a normal constituent of an organ or tissue, as well as a tissue repair mechanism. Fibrosis, or the formation or development of excess fibrous connective tissue in an organ or tissue as a reparative or reactive process, occurs in a variety of diverse tissues when this process is dysregulated by impaired re-epithelialization in association with myofibroblast activation (Tomasek et al. Nat Rev Mol Cell Biol 3, 349-363 (2002), Thannickal et al Annu Rev Med 55, 395-417 (2004), Hinz et al. Am J Pathol 170, 1807-1816 (2007), herein incorporated by reference in their entireties). Mechanisms of myofibroblast differentiation and activation involve the coordinated actions of transforming growth factor-β1 (TGF-(β1), matrix signaling, and biomechanical tension (Desmouliere et al. J Cell Biol 122, 103-111 (1993), Serini et al. J Cell Biol 142, 873-881 (1998), Hinz et al. Am J Pathol 159, 1009-1020 (2001), herein incorporated by reference in their entireties). Myofibroblast differentiation by TGF-β1 is associated with the activation of a flavoenzyme that generates extracellular hydrogen peroxide (H₂O₂) (Thannickal & Fanburg. J Biol Chem 270, 30334-30338 (1995), Thannickal et al. J Biol Chem 278, 12384-12389 (2003), herein incorporated by reference in their entireties). To date, treatment of fibrotic diseases and condition has been very limited. What are needed are better compositions and methods for diagnosing, characterizing, identifying treatments for, and treating fibrotic diseases and conditions (e.g. pulmonary fibrosis).

### SUMMARY OF THE INVENTION

The present invention relates to biomarkers, therapeutic targets, and therapeutic agents for treating and diagnosing fibrotic disorders. In particular, the present invention relates to diagnosis, drug screening, and therapeutic targeting of NOX4 biomarkers of pulmonary fibrosis and other fibrotic diseases and conditions. The compositions and methods of the present invention find use in diagnostic, therapeutic, research, and drug screening applications. The present invention further provides assay for identifying, characterizing, and testing therapeutic agents that find use in treating fibrosis.

In some embodiments, the present invention provides a method of treating fibrosis, comprising: inhibiting NOX4 in a subject. In some embodiments, treating fibrosis comprises preventing fibrosis in a subject at risk for fibrosis. In some embodiments, treating fibrosis comprises treating fibrosis in a subject suffering from fibrosis. In some embodiments, inhibiting NOX4 comprises inhibiting expression of the NOX4 gene. In some embodiments, inhibiting NOX4 comprises inhibiting NOX4 enzymatic activity. In some embodiments, the inhibiting NOX4 comprises administering NOX4-inhibiting compounds to a subject. In some embodiments, inhibiting NOX4 comprises administering siRNA to a subject. In some embodiments, inhibiting NOX4 comprises administering one or more small molecule drugs to a subject.

In some embodiments, the present invention provides a method of treating or preventing pulmonary fibrosis in a subject, comprising inhibiting NOX4 in the subject. In some embodiments, the subject is at risk for pulmonary fibrosis. In some embodiments, the subject suffers from pulmonary fibrosis. In some embodiments, inhibiting NOX4 comprises inhibiting expression of the NOX4 gene. In some embodiments, inhibiting NOX4 comprises inhibiting NOX4 enzymatic activity. In some embodiments, inhibiting NOX4 comprises administering one or more NOX4-inhibiting agents to the subject. In some embodiments, inhibiting NOX4 comprises administering siRNA to the subject. In some embodiments, siRNA administered to a subject comprise one or more of SEQ ID NOs:1-10. In some embodiments, inhibiting NOX4 comprises administering one or more small molecule drugs to the subject. In some embodiments, inhibiting NOX4 comprises administering antibodies to the subject. In some embodiments, the pulmonary fibrosis is unresponsive to one or more other standard treatments.

In some embodiments, the present invention provides a method of detecting or characterizing fibrosis in a subject, comprising detecting one or more biomarkers of fibrosis in a subject, at least one of the markers being a NOX4 biomarker. In some embodiments, detecting fibrosis in a subject determines or assists in selecting a future course of treatment for the subject. In some embodiments, the present invention provides a method of detecting pulmonary fibrosis in a subject, comprising detecting one or more biomarkers of pulmonary fibrosis in the subject, at least one of the biomarkers being a NOX4 biomarker.

In some embodiments, the present invention provides a method of screening candidate compounds for effectiveness in treating pulmonary fibrosis comprising: (a) providing (i) one or more candidate compounds and (ii) one or more cells expressing biomarkers of pulmonary fibrosis, (b) administering one or more of the compounds to one or more of the cells, (c) detecting the effect of the compounds on the biomarkers, and (d) comparing the effect to cells not administered the compounds. In some embodiments, one or more of the biomarkers comprises a NOX4 biomarker. In some embodiments, the detecting the effect of the compounds on the biomarkers comprises detecting the expression of the biomarkers. In some embodiments, detecting the effect of the compounds on the biomarkers comprises detecting the activity of the biomarkers. In some embodiments, the present invention provides a method of screening candidate compounds for effectiveness in treating pulmonary fibrosis comprising (a) providing (i) one or more candidate compounds; and (ii) one or more cells expressing biomarkers of pulmonary fibrosis, wherein one of the biomarkers is NOX4; (b) administering one or more of the compounds to one or more of the cells; (c) detecting the effect of the compounds on the biomarkers; and (d) comparing the effect to cells not administered the compounds.

### DESCRIPTION OF THE DRAWINGS

FIG. 1 shows identification of NOX4 as the enzymatic source of extracellular H₂O₂ production by myofibroblasts and its role in mediating myofibroblast differentiation and contractility: (a) RNA was isolated from human fetal lung mesenchymal cells (hFLMCs) treated with/without TGF-β1 (2 ng/ml) for 18 h and analyzed by Affymetrix (U133A) microarray for members of the NOX/DUOX gene family; (b) hFLMCs were treated with/without TGF-β1 (2 ng/ml) for the times indicated and cell lysates subjected to SDS-PAGE and Western immunoblotting for NOX4 and GAPDH; (c) effect of NOX4 siRNA (duplex 4) on extracellular release of H₂O₂ by hFLMCs treated with/without TGF-β1 (2 ng/ml for 16 h); (d) hFLMCs were pretreated with pharmacologic inhibitors against ALK5 receptor kinase (SB431542; 1 μM), MEK (PD98059; 20 μM), p38 MAPK (SB203580; 6 1 μM), JNK (SP600125; 100 nM), and then stimulated with TGF-β1 (2 ng/ml×16 h) prior to measurement of extracellular H₂O₂ release; (e) effect of SMAD3 siRNA knockdown on TGF-β1-induced NOX4 expression in hFLMCs, as determined by Western immunoblotting; (f) Effect of siRNA mediated knockdown of SMAD3 on extracellular H₂O₂ production stimulated by TGF-β1 (2 ng/ml×16 h) in hFLMCs; (g) hFLMCs in 3-D collagen matrix were stimulated with/without TGF-β1 (2 ng/ml×16 h) in the presence/absence of catalase (750 U/ml) and effects on α-smooth muscle actin (α-SMA), fibronectin, and β-actin were determined by Western immunoblotting; (h) effect of siRNA-mediated silencing of NOX4 in 3D-collagen matrix embedded hFLMCs on cellular expression of α-SMA, fibronectin, and procollagen-1 treated with/without TGF-β1 (2.5 ng/ml×72 h), as determined by Western immunoblotting; (i,j) effect of exogenous catalase (750 U/ml) (i), and siRNA-mediated NOX4 silencing (j) on TGF-β1-induced contractility in 3D-collagen matrices.

FIG. 2 shows regulation of NOX4 by TGF-β1 in human fetal lung mesenchymal cells (hFLMCs) and IPF mesenchymal cells (IPF-MCs): (a) hFLMCs were treated with/without TGF-β1 (2 ng/ml) for 16 h and NOX4 mRNA determined by RT-PCR. (b) hFLMCs were treated with/without TGF-β1 (2 ng/ml) for the times indicated and cell lysates analyzed by Western immunoblotting for NOX4; blots were stripped and re-probed for GAPDH. Bars represent densitometric ratios of NOX4:GAPDH; (c) hFLMCs were transfected with individual siRNA duplexes for 4 days and then treated with/without TGF-β1 (2 ng/ml) for 24 h; expression of NOX4 and GAPDH were determined by Western immunoblotting; (d) IPF-MCs were transfected with non-targeting (control) siRNA or SMAD3 siRNA and treated with/without TGF-β1 (2 ng/ml) for 48 h, followed by Western immunoblot analyses for the proteins indicated.

FIG. 3 shows NOX4 is expressed in lungs of human subjects with idiopathic pulmonary fibrosis (IPF) and mediates H₂O₂ production, myofibroblast differentiation, and serum-stimulated proliferation of IPF-derived mesenchymal cells: (a) immunohistochemical staining demonstrating expression of NOX4 in myofibroblastic foci in lungs of a representative human subject with IPF (length bar=100 μm); (b-i) Mesenchymal cells isolated from IPF lung tissues (IPF-MCs) by explant tissue culture and analyzed at passage: (b) IPF-MCs were transfected with nontargeting (control) siRNA or NOX4 siRNA and treated with/without TGF-β1 (2 ng/ml) for 16 h and analyzed for NOX4 protein (inset) and extracellular H₂O₂ production; (c-f) the effect of siRNA knockdown of NOX4 in IPF-MCs with/without TGF-β1 (2 ng/ml) on the expression of α-SMA mRNA (c) and protein (f); fibronectin mRNA (d) and protein (f); and NOX4 mRNA (e) and protein (f), as determined by real-time PCR (at 24 h) and Western immunoblotting (at 48 h); (g) Control (nontargeting) and NOX4 siRNA transfected IPF-MCs were treated with/without TGF-β1 (2 ng/ml) for 48 h and conditioned culture media was collected and analyzed for acid-soluble collagen using the Sircol assay; (h,i) the effect of siRNA knockdown of NOX4 on proliferation of IPF-MCs treated with/without serum was determined at 24 h by BrdU incorporation assay (h) and at 48 h by assessment of cell numbers using a coulter counter (i).

FIG. 4 shows NOX4 is induced during the fibrogenic phase of bleomycin-induced lung injury in mice and inhibition of NOX4 expression/activity attenuates pulmonary fibrosis: (a) C57BL/6 mice were subjected to acute lung injury by airway (intra-tracheal) administration of bleomycin or saline/control on day 0, following bleomycin injury, mice were euthanized at the indicated time intervals, whole lungs were harvested, and tissue homogenates analyzed by SDSPAGE and Western immunoblotting for NOX4, NOX2, and β-actin; (b-e) NOX4 siRNA or a nontargeting control siRNA was instilled directly down the trachea of mice at the time of bleomycin injury (day 0), and lungs were analyzed on day 14 or 21: (b) NOX4 expression on day 21 was determined by Western immunoblotting of whole lung homogenates; (c) fibrosis was assessed on day 14 by H & E staining and Masson's trichrome blue staining for collagen (top panels); NOX4 and α-SMA expression were assessed by immunohistochemical (IHC) analysis (bottom panels); length bar=100 μm; (d,e) Whole lung homogenates were analyzed on day 21 for hydroxyproline content (d), and on day 14 for acid-soluble collagen using the Sircol assay (e); (f) C57BL/6 mice were administered intra-tracheal (IT) bleomycin on day 0, diphenyleneiodonium (DPI; 1.6 mg/kg) or vehicle control was administered by daily intraperitoneal (IP) injections starting on day 7 for 14 days, whole lung homogenates were analyzed for acid-soluble collagen using the Sircol assay on day 21.

FIG. 5 shows validation of the efficacy of NOX4 siRNA in ex vivo culture of murine lung mesenchymal cells and the in vivo anti-fibrotic effects of pharmacologic inhibition of NOX/flavoenzyme activity during the post-inflammatory phase of bleomycin-induced lung injury in mice: (a) primary lung mesenchymal cells isolated from 4-6 week old C57BL/6 mice were transfected with nontargeting siRNA or NOX4 siRNA and treated with/without TGF-β1 (2 ng/ml) for 24 h. RNA was extracted and expression of NOX4 mRNA analyzed by real-time PCR; (b) C57BL/6 mice were administered intra-tracheal (IT) bleomycin on day 0. Diphenyleneiodonium (DPI; 1.6 mg/kg) or vehicle control was administered by daily intraperitoneal (IP) injections starting on day 7 for 14 days, fibrosis was assessed on day 21 by H & E staining and Masson's trichrome blue staining for collagen (top panels), NOX4 and α-SMA expression were assessed by immunohistochemical (IHC) analysis (bottom panels); length bar=100 nm.

FIG. 6 shows RNAi-mediated knockdown of NOX4 attenuates fibrosis in mice subjected to fluorescein isothiocyanate-induced lung injury; C57BL/6 mice were administered intratracheal fluorescein isothiocyanate (FITC) or saline/control on day 0 with nontargeting control siRNA or NOX4 siRNA and lung tissues were analyzed on day 14 or 21: (a) NOX4 expression on day 21 was determined by Western immunoblotting of whole lung homogenates; (b) fibrosis was assessed by H & E staining and Masson's trichrome blue staining for collagen; length bar=100 nm; (c,d) Whole lung homogenates were analyzed on day 14 for acid-soluble collagen using the Sircol assay (c), and on day 21 for hydroxyproline content (d).

### DEFINITIONS

As used herein, the term “fibrosis” may refer to any condition, disorder, state, and/or disease involving excess fibrous connective tissue in an organ or tissue. The term “fibrosis” may include, but is not limited to pulmonary (lung) fibrosis, cystic fibrosis (e.g. of the pancreas and/or lungs), injection fibrosis (e.g. as a complication of intramuscular injections, especially in children), endomyocardial fibrosis, idiopathic pulmonary fibrosis of the lung, mediastinal fibrosis, myelofibrosis, retroperitoneal fibrosis, progressive massive fibrosis (e.g. a complication of coal workers' pneumoconiosis), nephrogenic systemic fibrosis, complication of certain mellitus, etc.

As used herein, the term “subject” refers to any animal (e.g., a mammal), including, but not limited to, humans, non-human primates, bovines, equines, porcines, canines, felines, rodents, and the like. Typically, the term “patient” is used interchangeably with the term “subject” herein in reference to a human subject.

As used herein, the term “subject suspected of having fibrosis” refers to a subject that presents one or more symptoms indicative of fibrosis or is being screened for fibrosis (e.g., during a physical exam). A subject suspected of having fibrosis may also have one or more risk factors. A subject suspected of having fibrosis has generally not been tested for fibrosis. However, a “subject suspected of having fibrosis” encompasses an individual who has received a preliminary diagnosis but for whom a confirmatory test has not been done. A “subject suspected of having fibrosis” is sometimes subsequently diagnosed with fibrosis and is sometimes found to not have fibrosis.

As used herein, the term “subject diagnosed with fibrosis” refers to a subject who has been tested and found to have fibrosis. Fibrosis may be diagnosed using any suitable method, including but not limited to, the diagnostic methods of the present invention.

As used herein, the term “initial diagnosis” refers to a test result of initial fibrosis diagnosis that reveals the presence or absence of fibrosis. An initial diagnosis does not include information about the extent of fibrosis.

As used herein, the term “subject at risk for fibrosis” refers to a subject with one or more risk factors for developing fibrosis. Risk factors include, but are not limited to, genetic predisposition, environmental exposure, and lifestyle.

As used herein, the term “characterizing fibrosis in subject” refers to the identification of one or more properties of fibrosis in a subject (e.g. severity, degree of advancement, etc.). Fibrosis may be characterized by the identification of one or more markers (e.g., NOX4) of the present invention.

As used herein, the term “reagent(s) capable of specifically detecting biomarker expression” refers to reagents used to detect the expression of biomarkers (e.g., NOX4 and/or biomarkers described herein). Examples of suitable reagents include but are not limited to, nucleic acid probes capable of specifically hybridizing to mRNA or cDNA, and antibodies (e.g., monoclonal antibodies).

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “providing a prognosis” refers to providing information regarding the impact of the presence of fibrosis (e.g., as determined by the diagnostic methods of the present invention) on a subject's future health.

As used herein, the term “non-human animals” refers to all non-human animals including, but are not limited to, vertebrates such as rodents, non-human primates, ovines, bovines, ruminants, lagomorphs, porcines, caprines, equines, canines, felines, etc.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment is retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “transgene” refers to a heterologous gene that is integrated into the genome of an organism (e.g., a non-human animal) and that is transmitted to progeny of the organism during sexual reproduction.

As used herein, the term “transgenic organism” refers to an organism (e.g., a non-human animal) that has a transgene integrated into its genome and that transmits the transgene to its progeny during sexual reproduction.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., fibrosis). Test compounds comprise both known and potential therapeutic compounds. A test compound can be determined to be therapeutic by screening using the screening methods of the present invention.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present invention.

### DETAILED DESCRIPTION OF THE INVENTION

The present invention relates to biomarkers, therapeutic targets, and therapeutic agents for treating and diagnosing fibrotic disorders. In particular, the present invention relates to diagnosis, drug screening, and therapeutic targeting of NOX4 biomarkers ((Genbank Accession Number BC138918 (Mus musculus); Genbank Accession Number BC051371 (Homo sapiens); etc.)) of pulmonary fibrosis and other fibrotic diseases and conditions. The compositions and methods of the present invention find use in diagnostic, therapeutic, research, and drug screening applications. The present invention further provides assay for identifying, characterizing, and testing therapeutic agents that find use in treating fibrosis.

A homolog of the nicotinamide adenine dinucleotide phosphate (reduced form; NADPH) oxidase (NOX) family of oxidoreductases, NOX4, has been implicated in the differentiation of cardiac fibroblasts to myofibroblasts (Cucoranu et al. Circ Res 97, 900-907 (2005), herein incorporated by reference in its entirety).

The NOX enzyme family, which catalyze the reduction of O₂ to form reactive oxygen species (ROS), emerged during the evolutionary transition from unicellular to multicellular eukaryotes (Kawahara et al. BMC Evol Biol 7, 109 (2007), Bedard et al. Biochimie 89, 1107-1112 (2007), herein incorporated by reference in their entireties). With the increased number and diversity of NOX family enzymes in mammalian species, their biological functions and their roles in disease pathogenesis are yet to be elucidated (Lambeth. Nat. Rev Immunol 4, 181-189 (2004)., Bedard & Krause. Physiol Rev 87, 245-313 (2007), herein incorporated by reference in their entireties). The best established physiologic roles for the NOX gene family are in host defense against pathogen invasion in almost all species studied, including plants (Geiszt & Leto. J Biol Chem 279, 51715-51718 (2004), Levine et al. Cell 79, 583-593 (1994), herein incorporated by reference in their entireties). The prototypical member of this family, NOX2 (also known as gp91phox), is critical for defense against infectious microbes in mammals (Ahluwalia et al. Nature 427, 853-858 (2004), Quie et al. J Clin Invest 46, 668-679 (1967), herein incorporated by reference in their entireties). Additionally, the DUOX homolog in Drosophila has recently been shown to function in innate immunity of the gastrointestinal tract (Ha et al. Science 310, 847-850 (2005), herein incorporated by reference in its entirety).

Experiments conducted during the development of embodiments of the present invention identified that NOX4 is expressed or upregulated during fibrogenesis and/or fibrosis. In some embodiments, NOX4 is a biomarker for fibrosis and/or fibrogenisis.

Although an understanding of the mechanism is not necessary to practice the present invention and the present invention is not limited to any particular mechanism of action, in some embodiments, it is contemplated that transforming growth factor-β1 (TGF-β1) induces NOX4 expression in lung mesenchymal cells by a SMAD3-dependent mechanism; NOX4-dependent generation of hydrogen peroxide (H₂O₂) then results in myofibroblast differentiation, extracellular matrix (ECM) production, and contractility. Thereby, NOX4 expression, induced by TGF-β1 causes fibrogenisis and fibrosis. In some embodiments, monitoring expression and/or activity of NOX4 in cells, samples, or subjects provides for monitoring fibrogenisis and/or fibrosis. In some embodiments, targeting NOX4 expression or activity with inhibitors provides for inhibiting fibrogenesis and/or treating fibrosis. In some embodiments, monitoring the effects of agents (e.g. compounds, therapeutics, drugs, RNAi, etc.) on NOX4 expression or activity provides for screening for inhibitors or activators of fibrogenesis and/or therapeutics for fibrosis.

In some embodiments, the present invention provides markers which are differentially expressed in cells (e.g. myofibroblasts) engaged in fibrogenesis compared to non-fibrogenic cells, non-myofibroblasts, or control cells. In some embodiments, the present invention provides markers which are differentially expressed in fibrosis (e.g. pulmonary fibrosis) compared to non-fibrogenic cells or control cells. Such markers find use in the diagnosis and characterization and alteration (e.g., therapeutic targeting) of various fibrogenic conditions (e.g. fibrosis (e.g. pulmonary fibrosis)).

In some embodiments, the present invention provides compositions and methods for treating and/or preventing one or more forms of fibrosis (e.g. pulmonary fibrosis). In some embodiments, the present invention provides compositions and methods for treating and/or preventing pulmonary fibrosis. In some embodiments, the present invention treats and/or prevents fibrosis (e.g. pulmonary fibrosis) by inhibiting one or more biomarkers of fibrosis (e.g. pulmonary fibrosis) and/or fibrogenesis. In some embodiments, the present invention provides compositions and method for inhibiting NOX4 and/or additional biomarkers of fibrosis (e.g. pulmonary fibrosis). In some embodiments, fibrosis (e.g. pulmonary fibrosis) is treated through the inhibition of NOX4 activity or expression. In some embodiments, NOX4 is inhibited by administration of a NOX4-inhibiting agent. In some embodiments, a NOX4 inhibiting agent comprises any compound, protein (e.g. antibody), peptide, nucleic acid (e.g. siRNA), small molecule, macromolecule, etc. capable of inhibiting NOX4 expression and/or activity. In some embodiments, the present invention provides compositions to treat and/or prevent pulmonary fibrosis by inhibiting NOX4 activity and/or expression. In some embodiments, the present invention provides compositions to treat and/or prevent pulmonary fibrosis by providing a competing functionality to NOX4 (e.g. clearing ROS from a cell or subject).

In some embodiments, the present invention provides compositions and methods for characterizing agents for treating one or more forms of fibrosis (e.g. pulmonary fibrosis). For example, in some embodiments, the method comprises exposing an organism, tissue, or cell to an agent and assessing a change in a NOX4 (and/or other marker) biological activity or expression. In some embodiments, the organism, tissue, or cell comprises a heterologous NOX4 gene (or other marker). In some embodiments, the organism, tissue, or cell does not normally comprise the marker gene. In some embodiments, a change in biological activity is the effect of a change in marker expression (mRNA or protein). In some embodiments, a change in biological activity is a change in cell function. In some embodiments, a change in biological activity is a change in organism function (e.g., tissue health, signs or symptoms of disease, etc.).

In some embodiments, the present invention provides compositions and methods for characterizing agents for altering (e.g. reducing or increasing) fibrogenisis. In some embodiments, compositions or methods for increasing expression or activity of NOX4, increases the rate and/or amount of fibrogenesis in a sample or subject. In some embodiments, compositions or methods for decreasing expression or activity of NOX4, decreases the rate and/or amount of fibrogenesis in a sample or subject. In some embodiments, methods of the present invention comprise detecting the presence of, the absence of, or amount of, NOX4 expression or activity in a cell, subject, or sample.

In some embodiments, the present invention provides kits for the detection and characterization of fibrosis. In some embodiments, the kits contain reagents for detecting biomarkers described herein (e.g. NOX4) and/or antibodies specific for fibrosis biomarkers, in addition to detection reagents and buffers. In other embodiments, the kits contain reagents specific for the detection of fibrosis biomarker mRNA, SNPs, cDNA (e.g., oligonucleotide probes or primers), etc. In preferred embodiments, the kits contain all of the components useful, sufficient, or necessary to perform a detection assay, including all controls, directions for performing assays, and any necessary software for analysis and presentation of results.

In some embodiments, the expression of mRNA and/or proteins associated with biomarkers (e.g. NOX4 or other biomarkers of fibrosis or fibrogenesis) of the present invention are determined. In some embodiments, the presence or absence of biomarkers is correlated with mRNA and/or protein expression.

In some embodiments, gene silencing (e.g., via siRNA, antisense, or other RNAi approaches) is utilized to alter expression of genes associated with biomarkers described herein. In some embodiments, gene silencing is utilized to alter expression of NOX4. Exemplary siRNA target sequences for NOX4 include those provided in Table 1 (listing SEQ identifier, position, and sequence of each).

Table 1. siRNA target sequences for human NOX4
SEQ ID NO. 1 361 CAGAGTATCACTACCTCCACCAGAT
SEQ ID NO. 2 415 CCTCAGCATCTGTTCTTAACCTCAA
SEQ ID NO. 3 420 GCATCTGTTCTTAACCTCAACTGCA
SEQ ID NO. 4 514 GGAGAACCAGGAGATTGTTGGATAA
SEQ ID NO. 5 606 CATCTGGTGAATGCCCTCAACTTCT
SEQ ID NO. 6 623 CAACTTCTCAGTGAATTACAGTGAA
SEQ ID NO. 7 641 CAGTGAAGACTTTGTTGAACTGAAT
SEQ ID NO. 8 819 CATAACCTCTTCTTTGTCTTCTACA
SEQ ID NO. 9 868 GAGGGCTGCTGAAGTATCAAACTAA
SEQ ID NO. 10 869 AGGGCTGCTGAAGTATCAAACTAAT

Any number of other target sequences may be used. A variety of bioinformatic and experimental methods are available for selecting, testing, and optimizing such sequences.

In some embodiments, the present invention provides methods for detection of expression of markers (e.g., NOX4, fibrogenesis markers, fibrosis markers, etc.). In some embodiments, expression is measured directly (e.g., at the RNA or protein level). In some embodiments, expression is detected in tissue samples (e.g., biopsy tissue). In other embodiments, expression is detected in bodily fluids (e.g., including but not limited to, plasma, serum, whole blood, mucus, and urine). The present invention further provides panels and kits for the detection of markers. In some embodiments, the presence of a biomarker is used to provide a prognosis to a subject. The information provided is also used to direct the course of treatment. For example, if a subject is found to have a marker indicative of fibrosis or fibrogenesis, therapies can be initiated that are more likely to be effective.

The present invention is not limited to the markers described herein (e.g. NOX4). Any suitable marker that correlates with fibrogenesis and/or fibrosis can be utilized. Additional markers (e.g., NOX4 pathway members) are also contemplated to be within the scope of the present invention. Any suitable method can be utilized to identify and characterize biomarkers suitable for use in the methods of the present invention, including but not limited to, those described herein. For example, in some embodiments, markers identified as being up or down-regulated in fibrogenic cells using gene expression microarray methods are further characterized using tissue microarray, immunohistochemistry, Northern blot analysis, siRNA or antisense RNA inhibition, mutation analysis, investigation of expression with clinical outcome, as well as other methods disclosed herein.

In some embodiments, the present invention provides a panel for the analysis of a plurality of markers. The panel allows for the simultaneous analysis of multiple markers correlating with fibrogenesis and/or fibrosis. Depending on the subject, panels can be analyzed alone or in combination in order to provide the best possible characterization, diagnosis and/or prognosis. Markers for inclusion on a panel are selected by screening for their predictive value using any suitable method, including but not limited to, those described herein.

In some embodiments, RNA (or corresponding cDNA) is detected by hybridization to an oligonucleotide probe). A variety of hybridization assays using a variety of technologies for hybridization and detection are available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

In other embodiments, gene expression of biomarkers (e.g. NOX4 or other markers of fibrogenesis or fibrosis) is detected by measuring the expression of the corresponding protein or polypeptide. Protein expression can be detected by any suitable method. In some embodiments, proteins are detected by immunohistochemistry. In other embodiments, proteins are detected by their binding to an antibody raised against the protein. The generation of antibodies is well known in the art.

Antibody binding is detected by techniques known in the art (e.g., radioimmunoassay, ELISA (enzyme linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to biomarkers (e.g. NOX4 or other markers of fibrogenesis or fibrosis) is utilized.

In other embodiments, the immunoassay described in U.S. Pat. Nos. 5,599,677 and 5,672,480; each of which is herein incorporated by reference.

In some embodiments, the present invention provides drug screening assays (e.g., to screen for therapeutics, agents, and or drugs which promote fibrogenesis (e.g. pulmonary fibrosis), inhibit fibrogenesis (e.g. pulmonary fibrosis), treat fibrosis (e.g. pulmonary fibrosis), etc.). The screening methods of the present invention utilize biomarkers (e.g. NOX4 or other markers of fibrosis and/or fibrogenesis) identified using the methods of the present invention. For example, in some embodiments, the present invention provides methods of screening for compounds that alter (e.g., increase or decrease) the expression of biomarker genes (e.g. NOX4 or other markers of fibrosis and/or fibrogenesis). In some embodiments, candidate compounds are antisense agents or siRNA agents (e.g., oligonucleotides) directed against biomarkers. In other embodiments, candidate compounds are antibodies that specifically bind to a biomarker of the present invention (e.g. NOX4 or other markers of fibrosis and/or fibrogenesis). In certain embodiments, libraries of compounds of small molecules are screened using the methods described herein. In some embodiments, high through-put methods of compound screening are utilized.

In one screening method, candidate compounds are evaluated for their ability to alter biomarker expression by contacting a compound with a cell expressing a biomarker (e.g. NOX4 or other markers of fibrosis and/or fibrogenesis) and then assaying for the effect of the candidate compounds on expression. In some embodiments, the effect of candidate compounds on expression of a biomarker gene (e.g. NOX4 or other markers of fibrosis and/or fibrogenesis) is assayed by detecting the level of biomarker mRNA expressed by the cell. mRNA expression can be detected by any suitable method. In other embodiments, the effect of candidate compounds on expression of biomarker genes (e.g. NOX4 or other markers of fibrosis and/or fibrogenesis) is assayed by measuring the level of polypeptide encoded by the biomarkers. The level of polypeptide expressed can be measured using any suitable method, including but not limited to, those disclosed herein. In some embodiments, other changes in cell biology are detected.

Specifically, the present invention provides screening methods for identifying modulators, i.e., candidate or test compounds or agents (e.g., proteins, peptides, peptidomimetics, peptoids, small molecules or other drugs) which bind to, or alter the signaling or function associated with the biomarkers (e.g. NOX4 or other markers of fibrosis (e.g. pulmonary fibrosis) and/or fibrogenesis) of the present invention, have an inhibitory (or stimulatory) effect on, for example, biomarker expression or activity, or have a stimulatory or inhibitory effect on, for example, the expression or activity of a biomarker substrate. Compounds thus identified can be used to modulate the activity of target gene products (e.g., NOX4) either directly or indirectly in a therapeutic protocol, to elaborate the biological function of the target gene product, or to identify compounds that disrupt normal target gene interactions. In some embodiments, compounds which inhibit the activity or expression of biomarkers are useful in the treatment of fibrosis (e.g. pulmonary fibrosis).

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds can be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

This invention further pertains to novel agents identified by the above-described screening assays. Accordingly, it is within the scope of this invention to further use an agent identified as described herein (e.g., a biomarker modulating agent, an antisense biomarker nucleic acid molecule, a siRNA molecule, a biomarker specific antibody, or a biomarker-binding partner) in an appropriate animal model to determine the efficacy, toxicity, side effects, or mechanism of action, of treatment with such an agent. Furthermore, novel agents identified by the above-described screening assays can be, e.g., used for treatments (e.g. to treat a human patient for fibrosis (e.g. pulmonary fibrosis)).

In some embodiments, RNAi is utilized to inhibit fibrosis (e.g. pulmonary fibrosis) and/or fibrogenesis biomarkers (e.g. NOX 4 or others). RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, it is believed the sequence is delivered to an enzyme complex called RISC(RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments.

Chemically synthesized siRNAs have become powerful reagents for genome-wide analysis of mammalian gene function in cultured somatic cells. Beyond their value for validation of gene function, siRNAs also hold great potential as gene-specific therapeutic agents (Tuschl and Borkhardt, Molecular Intervent. 2002; 2(3):158-67, herein incorporated by reference).

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001;15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

An important factor in the design of siRNAs is the presence of accessible sites for siRNA binding. Bahoia et al., (J. Biol. Chem., 2003; 278: 15991-15997; herein incorporated by reference) describe the use of a type of DNA array called a scanning array to find accessible sites in mRNAs for designing effective siRNAs. Additional methods and concerns for selecting siRNAs are described for example, in WO 05054270, WO05038054A1, WO03070966A2, J Mol. Biol. 2005 May 13; 348(4):883-93, J Mol. Biol. 2005 May 13; 348(4):871-81, and Nucleic Acids Res. 2003 Aug. 1; 31(15):4417-24, each of which is herein incorporated by reference in its entirety. In addition, software (e.g., the MWG online siMAX siRNA design tool) is commercially or publicly available for use in the selection of siRNAs. In some embodiments, commercial services (e.g., those provided by Invitrogen, Carlsbad, Calif.) are utilized in the design of siRNA sequences.

In some embodiments, the present invention utilizes siRNA including blunt ends (See e.g., US20080200420, herein incorporated by reference in its entirety), overhangs (See e.g., US20080269147A1, herein incorporated by reference in its entirety), locked nucleic acids (See e.g., WO2008/006369, WO2008/043753, and WO2008/051306, each of which is herein incorporated by reference in its entirety). In some embodiments, siRNAs are delivered via gene expression or using bacteria (See e.g., Xiang et al., Nature 24: 6 (2006) and WO06066048, each of which is herein incorporated by reference in its entirety).

In other embodiments, shRNA techniques (See e.g., 20080025958, herein incorporated by reference in its entirety) are utilized. A small hairpin RNA or short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. shRNA is transcribed by RNA polymerase III.

In other embodiments, expression of fibrosis (e.g. pulmonary fibrosis) and/or fibrogenesis biomarkers (e.g. NOX 4 or others) is modulated using antisense compounds that specifically hybridize with one or more nucleic acids encoding biomarker protein (e.g. NOX 4 or other fibrosis and/or fibrogenesis biomarkers). The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of biomarker genes (e.g. NOX 4 or other fibrosis and/or fibrogenesis biomarkers). In the context of the present invention, “modulation” means either an increase (stimulation) or a decrease (inhibition) in the expression of a gene.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding a biomarker gene (e.g. NOX 4 or other fibrosis and/or fibrogenesis biomarkers). The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in PCT Publ. No. WO0198537A2, herein incorporated by reference.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

Specific examples of preferred antisense compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other preferred oligonucleotide mimetics, both the sugar and the internucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science 254:1497 (1991).

Most preferred embodiments of the invention are oligonucleotides with phosphorothioate backbones and oligonucleosides with heteroatom backbones, and in particular —CH₂, —NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂—, and —O—N(CH₃)—CH₂—CH₂—[wherein the native phosphodiester backbone is represented as—O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Hely. Chim. Acta 78:486 [1995]) i.e., an alkoxyalkoxy group. A further preferred modification includes 2′-dimethylaminooxyethoxy (i.e., a O(CH₂)₂ON(CH₃)₂ group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other preferred modifications include 2′-methoxy (2′-O—CH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2. ° C. and are presently preferred base substitutions, even more particularly when combined with 2′-O-methoxyethyl sugar modifications.

Another modification of the oligonucleotides of the present invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-5-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the antisense oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of the present invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNaseH is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the present invention may be formed as composite structures of two or more oligonucleotides, modified oligonucleotides, oligonucleosides and/or oligonucleotide mimetics as described above.

The present invention further provides pharmaceutical compositions (e.g., comprising a small molecule, antisense, antibody, or siRNA that targets the biomarkers (e.g. pulmonary fibrosis) biomarkers of the present invention (e.g. NOX4 or other biomarkers of fibrogenesis or fibrosis (e.g. pulmonary fibrosis))). The pharmaceutical compositions of the present invention can be administered in a number of ways depending upon whether local or systemic treatment is desired and upon the area to be treated. Administration can be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like can be necessary or desirable. Compositions and formulations for oral administration include powders or granules, suspensions or solutions in water or non aqueous media, capsules, sachets or tablets. Thickeners, flavoring agents, diluents, emulsifiers, dispersing aids or binders can be desirable. Compositions and formulations for parenteral, intrathecal or intraventricular administration can include sterile aqueous solutions that can also contain buffers, diluents and other suitable additives such as, but not limited to, penetration enhancers, carrier compounds and other pharmaceutically acceptable carriers or excipients. Pharmaceutical compositions of the present invention include, but are not limited to, solutions, emulsions, and liposome containing formulations. These compositions can be generated from a variety of components that include, but are not limited to, preformed liquids, self emulsifying solids and self emulsifying semisolids.

The pharmaceutical formulations of the present invention, which can conveniently be presented in unit dosage form, can be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product. The compositions of the present invention can be formulated into any of many possible dosage forms such as, but not limited to, tablets, capsules, liquid syrups, soft gels, suppositories, and enemas. The compositions of the present invention can also be formulated as suspensions in aqueous, non aqueous or mixed media. Aqueous suspensions can further contain substances that increase the viscosity of the suspension including, for example, sodium carboxymethylcellulose, sorbitol and/or dextran. The suspension can also contain stabilizers. In one embodiment of the present invention the pharmaceutical compositions can be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level can also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention can additionally contain other adjunct components conventionally found in pharmaceutical compositions. Thus, for example, the compositions can contain additional, compatible, pharmaceutically active materials such as, for example, antipruritics, astringents, local anesthetics or anti inflammatory agents, or can contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Certain embodiments of the invention provide pharmaceutical compositions containing (a) one or more compounds (e.g. antibody, small molecule, siRNA, anti-sense, etc.) that modulate the activity of a biomarker (e.g. NOX4 or other biomarkers of fibrogenesis and/or fibrosis (e.g. pulmonary fibrosis)) and (b) one or more other agents.

The present invention contemplates the generation of transgenic animals comprising an exogenous biomarker gene of the present invention (e.g. NOX4 or other biomarkers of fibrogenesis and/or fibrosis (e.g. pulmonary fibrosis)) or mutants and variants thereof (e.g., truncations or single nucleotide polymorphisms) or knock-outs thereof. In some embodiments, the transgenic animal displays an altered phenotype (e.g., increased or decreased presence of markers) as compared to wild-type animals. Methods for analyzing the presence or absence of such phenotypes include but are known to those in the art. In some embodiments, the transgenic animals further display increased or decreased fibrosis (e.g. pulmonary fibrosis) or fibrogenesis. The transgenic animals of the present invention find use in drug (e.g., fibrosis therapy) screens. In some embodiments, test compounds (e.g., a drug that is suspected of being useful to treat fibrosis) and control compounds (e.g., a placebo) are administered to the transgenic animals and the control animals and the effects evaluated. The transgenic animals can be generated via a variety of methods.

### EXPERIMENTAL

The following examples are provided in order to demonstrate and further illustrate certain embodiments and aspects of the present invention and are not to be construed as limiting the scope thereof.

### Example 1

### Materials and Methods

### Reagents

Porcine platelet-derived TGF-β1 was obtained from R&D Systems, Minneapolis, Minn.; protease inhibitor cocktail set III from Calbiochem, San Diego, Calif. Monoclonal antibodies to fibronectin (clone IST-4) and β-actin (clone AC-15) were obtained from Sigma, St. Louis, Mo.; monoclonal antibody to α-SMA (clone 1A4) from Dako, Carpinteria, Calif.; antibody to SMAD3 from Cell Signaling Technology, Danvers, Mass.; antibody to procollagen-I from Cederlane laboratories, Hornby, Ontario, Canada; and rabbit polyclonal antibody to GAPDH antibody from Abcam Inc., Cambridge, Mass. All other reagents were obtained from Sigma unless otherwise specified.

### Cell Culture

Human fetal lung mesenchymal cells (hFLMCs; IMR-90 cells) were obtained from Coriell Cell Repositories, Institute for Medical Research, Camden, N.J. Isolated primary mesenchymal cells were obtained from the lungs of C57BL/6 mice as previously described (Vittal et al. Am J Pathol 166, 367-375 (2005), herein incorporated by reference in its entirety). Primary lung mesenchymal cells isolated by explant cultures were obtained from lung tissues of human subjects with IPF (IPF-MCs) under an approved protocol by the Institutional Review Board at the University of Michigan. Informed consent was obtained from all individuals. All mesenchymal cells were cultured in DMEM (Life Technologies, Inc.) supplemented with 10% fetal calf serum (Hyclone Laboratories, Logan, Utah), 100 U/ml penicillin, 100 μg/ml streptomycin, and 1.25 μg/ml amphotericin B, and incubated cells at 37° C. in 5% CO2, 95% air.

### 3D-Collagen Gels

Collagen gels were reconstituted by mixing 1 part of 3 mg/ml neutralized rat tail collagen type 1 and two parts of cell suspension in serum-free media. Cell suspensions were seeded at a density of 200,000 cells per ml into 24-well tissue culture plates and allowed the gels to polymerize at 37° C. for 1 h before adding 1 ml of media. Gels were incubated overnight prior to treatments, and gently detached the edge of the gels from the walls of the well using a sterile spatula. Gels were photographed and gel area was measured using ImageJ (NIH) software.

### Murine Model of Bleomycin/FITC Lung Injury

C57BL/6J mice (6-8 weeks of age; Jackson Laboratories, Bar Harbor, Me.) were anesthetized with intraperitoneal injection of ketamine and xylazine. Intra-tracheal bleomycin (0.025 U) or FITC (4.67 mg/ml suspension in PBS) was administered to induce lung injury. The University of Michigan Committee on the Use and Care of Animals approved the animal protocols.

### RNA Interference

For in vitro RNAi, cells were transfected with single duplexes targeting NOX4 or non-targeting control siRNA (100 nM) using Lipofectamine-2000 reagent. For in vivo RNAi studies, NOX4 siRNA or control nontargeting siRNA (Dharmacon Inc.) were administered at a dose of ˜50 μg per mouse by intratracheal injection with bleomycin, FITC, or saline, in a total volume of 50 μl using a 26-gauge needle.

### Real-Time PCR

Total RNA was isolated from cells using the RNeasy® Mini Kit (Qiagen) and reverse transcribed using M-MLV RT First-Strand Synthesis (Invitrogen) as per manufacturers' protocols. Real-time PCR reactions were conducted for each cDNA sample in triplicate using SYBR® Green PCR Master Mix (Applied Biosystems) and gene specific primer pairs for α-actin, NOX4, α-SMA, and fibronectin (Supplementary Table 1). Reactions ran for 40 cycles (95° C. for 15 sec, 60° C. for 1 min) in a 7300 Real Time PCR System (Applied Biosystems, Foster City, Calif.). Semi-quantitative real-time PCR data was expressed for each target gene as 2-Ct Relative Quantitation (RQ) versus endogenous β-actin, with error bars representing the standard error of the mean for triplicate reactions. 2-way analysis of variance (ANOVA) was performed with Bonferroni post-test on grouped data.

### Gene Microarray Analyses

RNA isolates were hybridized on microarray Affymetrix U133A chips with 22976 probe pairs and the University of Michigan Microarray Core facility performed the statistical analyses.

### Western Immunoblotting

Cell lysates were prepared in RIPA buffer, subjected them to SDS-PAGE under reducing conditions and performed western immunoblotting as previously described (Horowitz et al. J Biol Chem 279, 1359-1367 (2004), herein incorporated by reference in its entirety).

### Measurement of H₂O₂ Production

Extracellular H₂O₂ release from cultured cells was assay as previously described (Thannickal & Fanburg. J Biol Chem 270, 30334-30338 (1995), herein incorporated by reference in its entirety).

### BrdU Incorporation Assay and Coulter Counting

Serum-starved cells were cultured in 96-well plates for 24 h followed by BrdU pulse in media with/without 10% serum for 24 hours. BrdU incorporation was measured using a kit from Calbiochem (Cat #QIA58). Cell counts were measured using a coulter counter (model ZM, Coulter Electronic, Hialeah, Fla.).

Sircol Assay for Collagen

Acid-soluble collagen was measured in cell culture supernatants or whole lung homogenates using the Sircol assay as previously described (Vittal et al. Am J Pathol 166, 367-375 (2005), herein incorporated by reference in its entirety).

### Hydroxyproline Content of Whole Lung

Murine whole lungs were homogenized in PBS, then acidified (by adding an equal volume of 12 N HCl), hydrolyzed (by heating at 120° C. for 24 h), and processed samples for hydroxyline measurements as previously described 29. Lung histology and immunohistochemical staining Paraffin embedded tissue sections were processed for lung histology and immunohistochemical staining as previously described described (Vittal et al. Am J Pathol 166, 367-375 (2005), herein incorporated by reference in its entirety).

### Example 2

### NOX4: Biomarker of Fibrosis

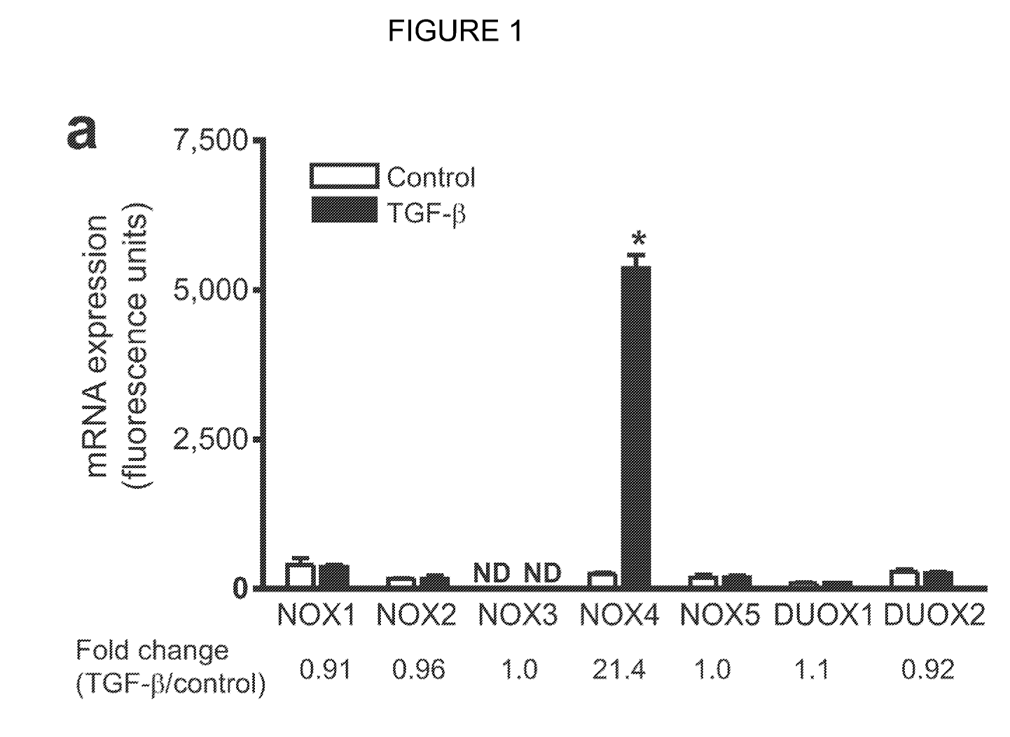

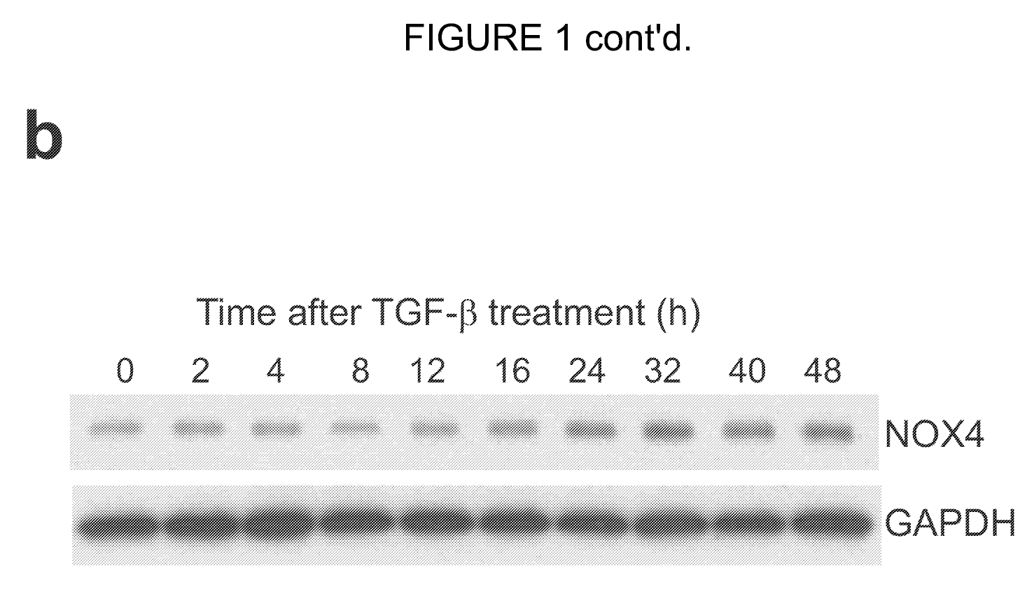

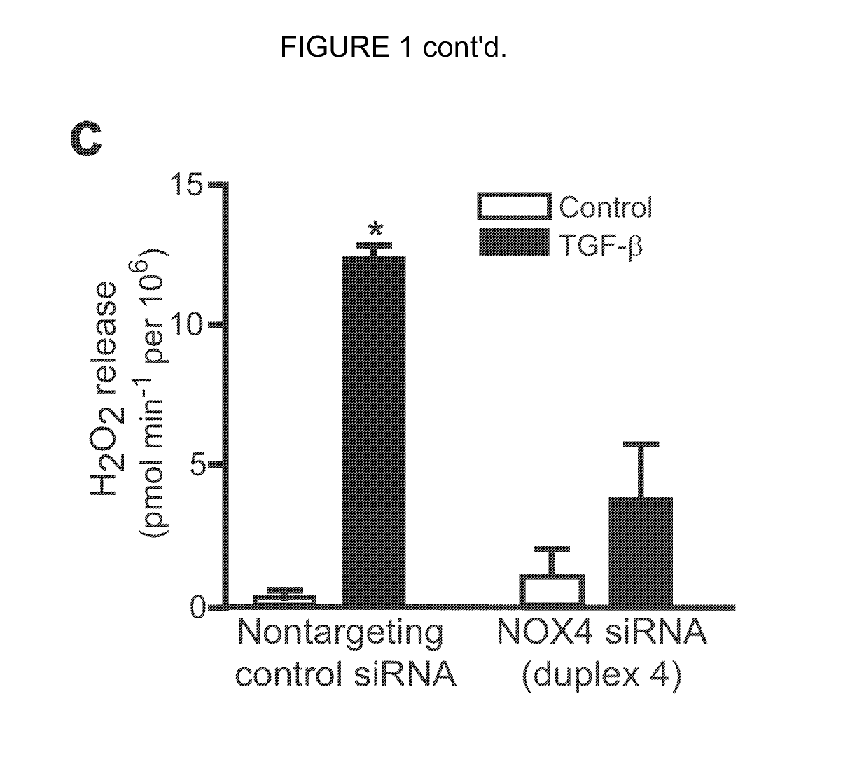

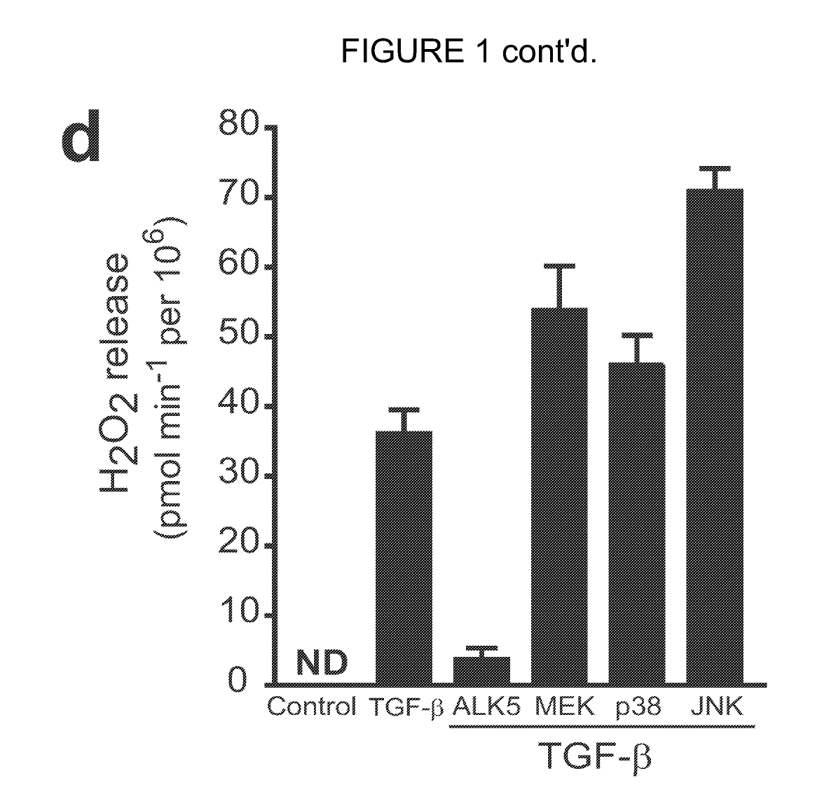

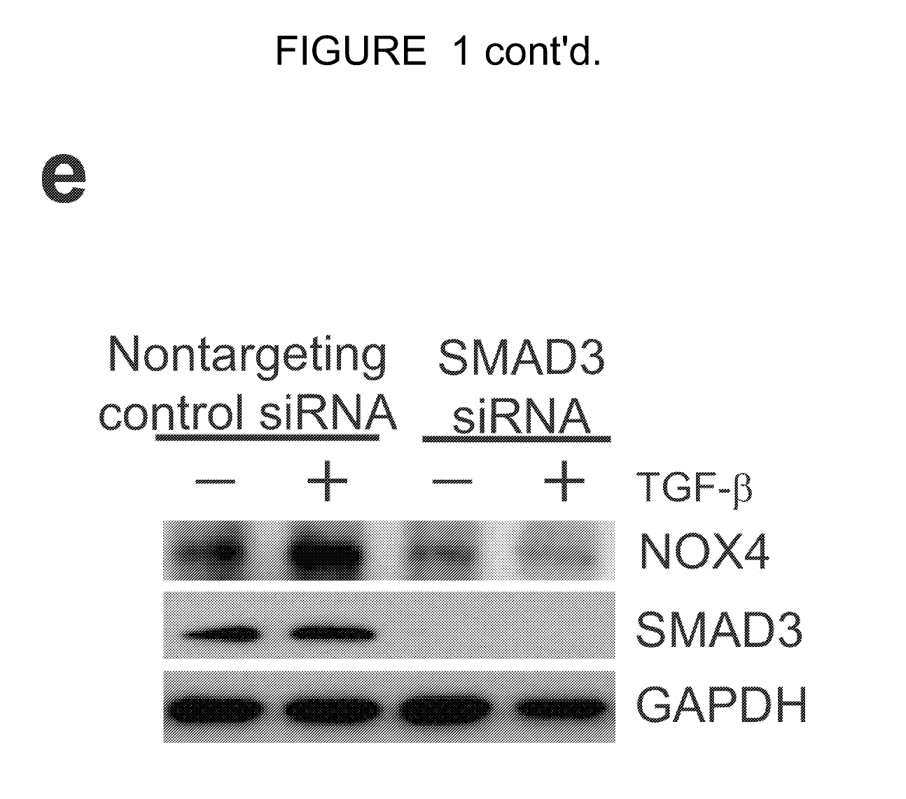

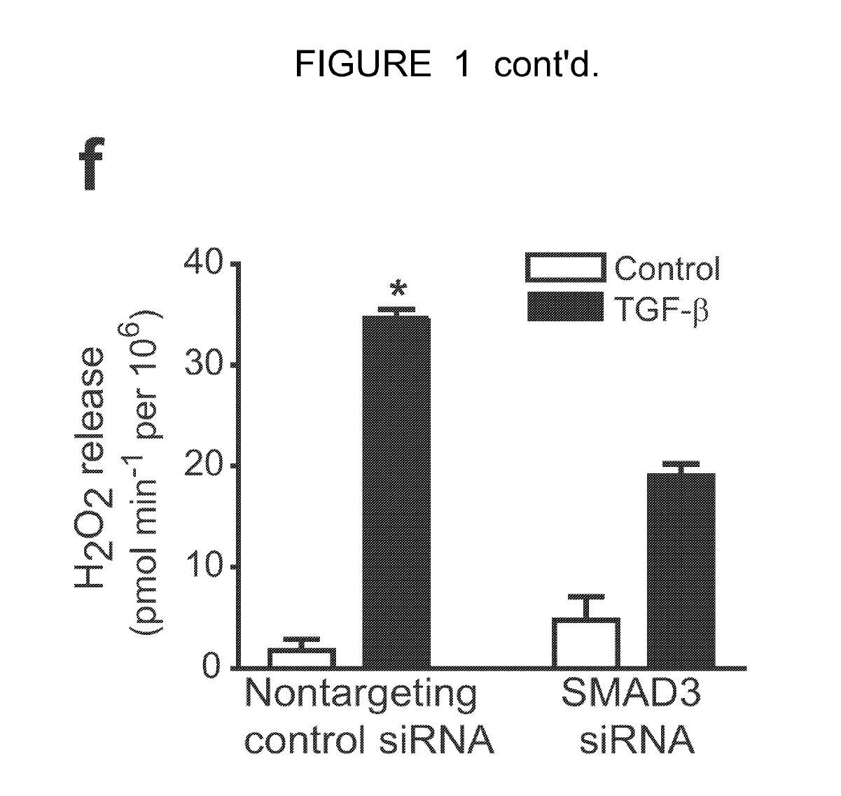

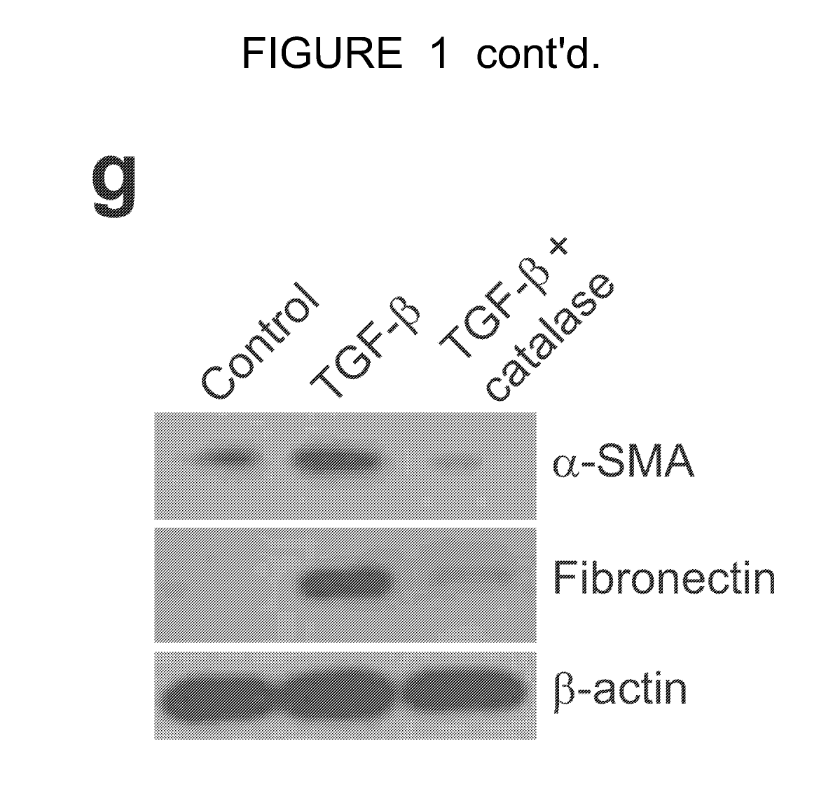

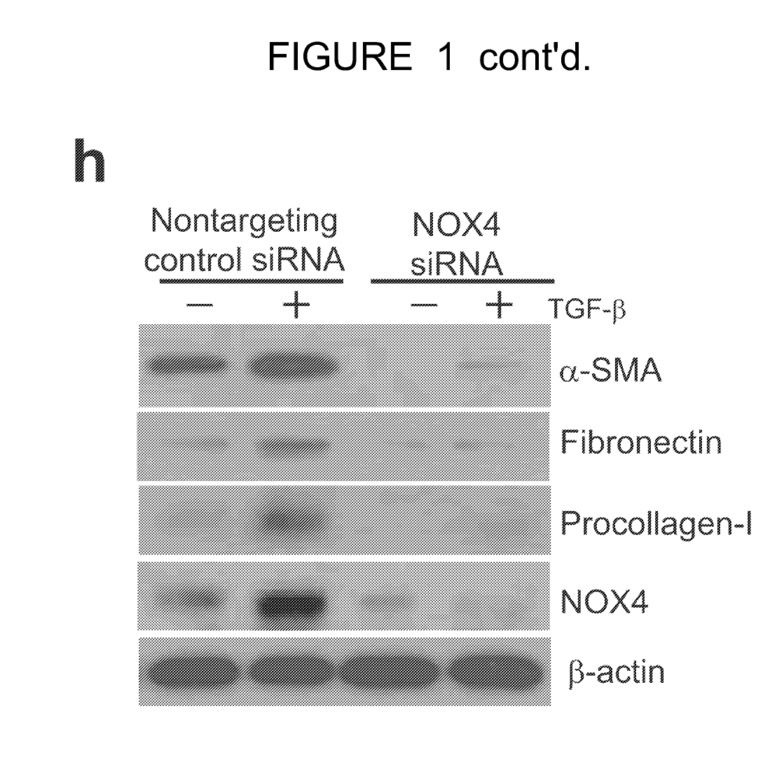

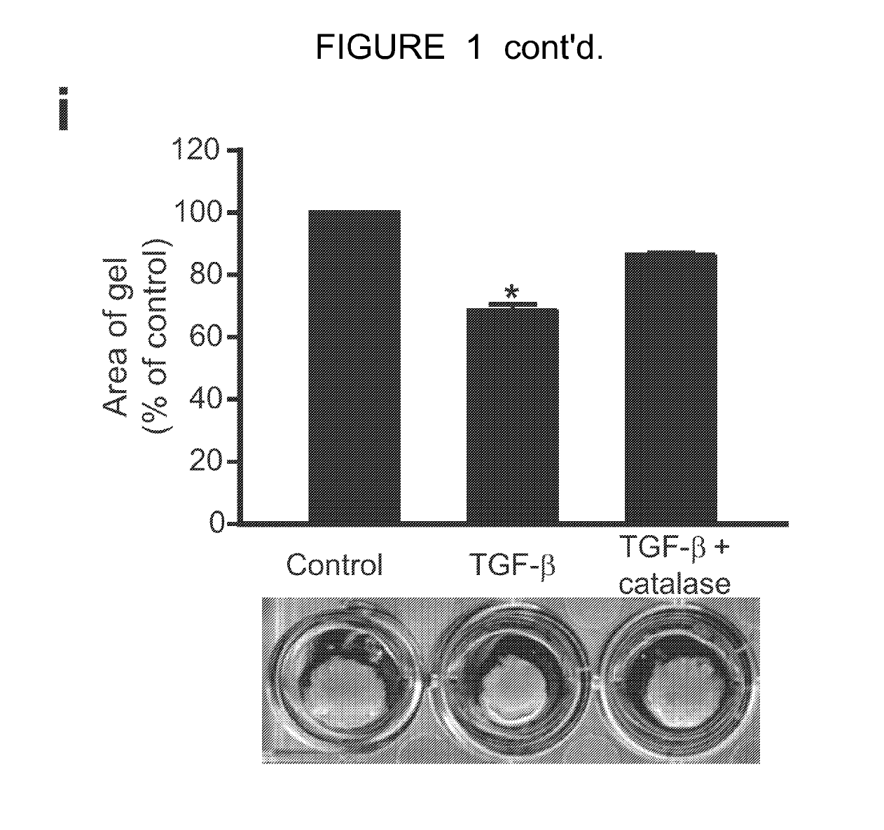

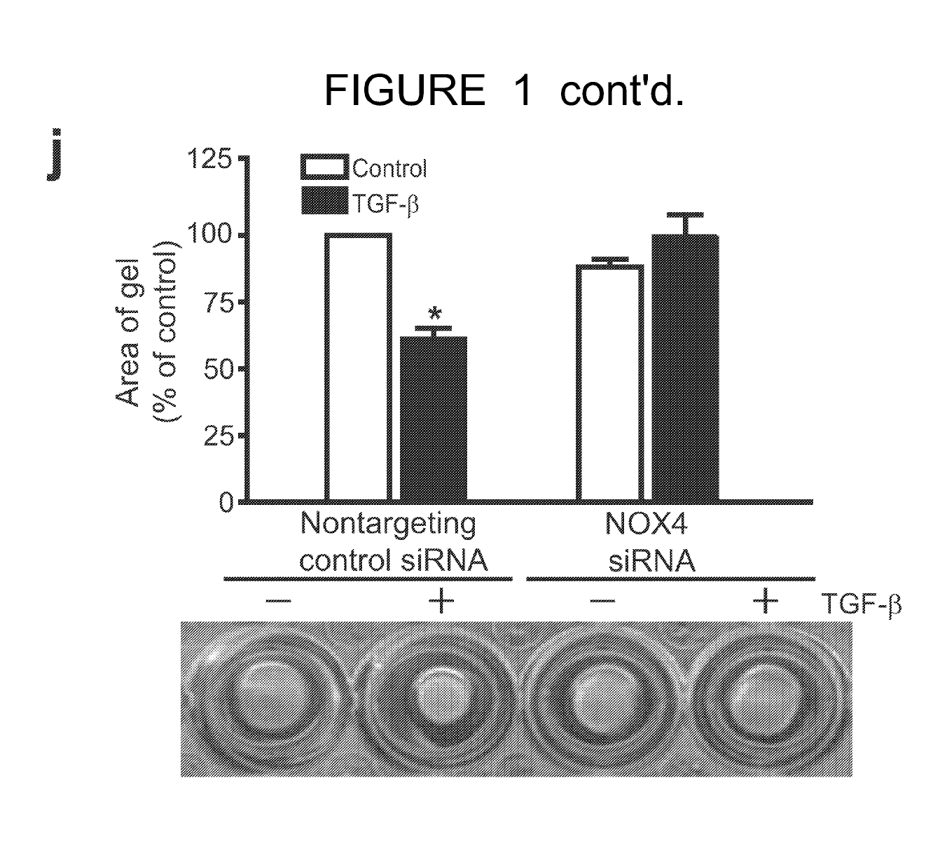

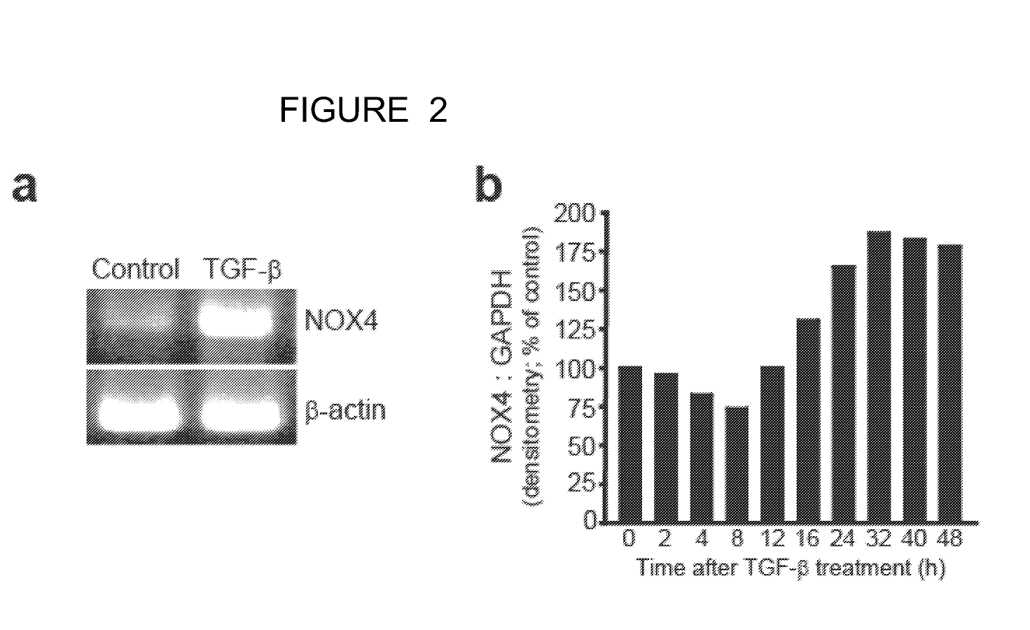

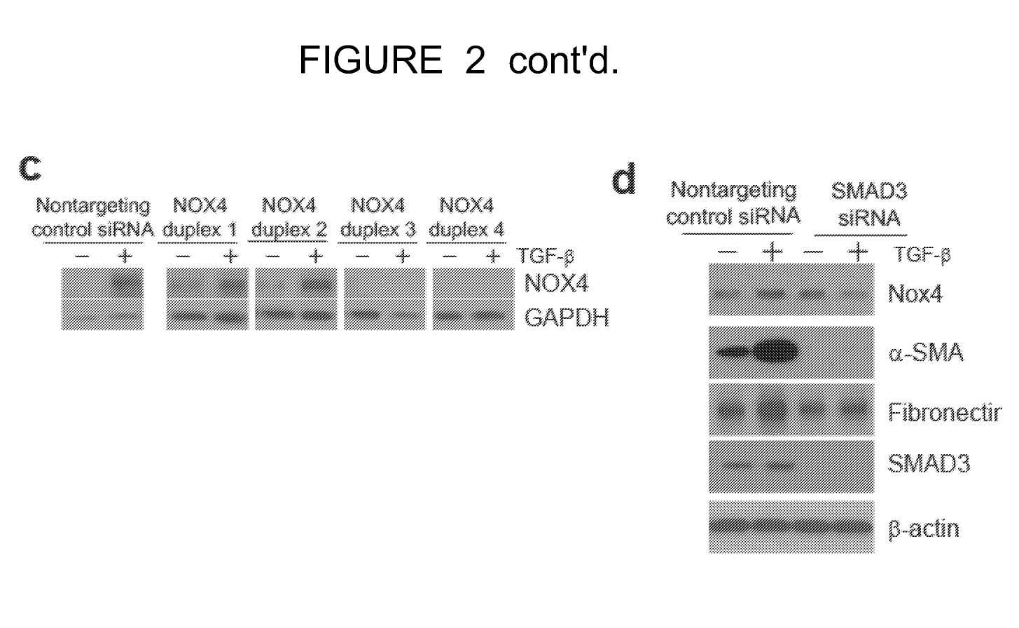

NOX4 has been implicated in the differentiation of cardiac fibroblasts to myofibroblasts (Cucoranu et al. Circ Res 97, 900-907 (2005), herein incorporated by reference in its entirety). Experiments performed during development of embodiments of the present invention identified NOX4 as one of the most highly induced genes by whole-genome Affymetrix analysis in human fetal lung mesenchymal cells (hFLMCs) stimulated with TGF-β1; other members of the NOX gene family were not affected at the mRNA level (SSE FIG. 1A). The upregulation of NOX4 mRNA by TGF-β1 was confirmed by RT-PCR (Supplementary FIG. 1a) and NOX4 protein expression was induced in a time-dependent manner (SEE FIGS. 1B and 2B). A RNA interference (RNAi) approach utilizing small interfering RNA (siRNA) targeting NOX4 was employed to define the specific role of NOX4. Two of four siRNA duplexes, duplex 3 and duplex 4, efficiently blocked NOX4 induction by TGF-β1 (SEE FIG. 2C). The NOX4 siRNA duplex 4 was utilized in subsequent in vitro studies to examine the role for NOX4 in myofibroblast differentiation and activation.

RNAi-mediated knockdown of NOX4 significantly inhibited TGF-β1-induced H₂O₂ production in hFLMCs (SEE FIG. 1C), indicating that NOX4 is the primary enzymatic source of extracellular H₂O₂ generation by TGF-β1-differentiated myofibroblasts. TGF-β1 signals via two heterodimeric transmembrane receptors, the type II and type I (ALK5) receptors. To define upstream mechanisms of TGF-β1-induced NOX4 induction and H₂O₂ generation in myofibroblasts, the effect of pharmacologic inhibitors of ALK5 and canonical MAPK pathways was tested. Only ALK5 inhibition attenuated the induction of H₂O₂ production by hFLMCs (SEE FIG. 1D). The ALK5 receptor is known to activate SMAD2 and SMAD3; however, pro-fibrotic effects TGF-β1/ALK5 signaling have been largely attributed to SMAD3 signaling (Bonniaud et al. J Immunol 173, 2099-2108 (2004), herein incorporated by reference in its entirety). An RNAi strategy was employed to determine if SMAD3 is required for NOX4 induction and H₂O₂ generation in hFLMCs; SMAD3 siRNA knockdown inhibited TGF-β1-induced NOX4 inducibility (SEE FIG. 1E) and H₂O₂ production (SEE FIG. 1F). A requirement for SMAD3 signaling in TGF-β1-induced NOX4 expression was observed in primary mesenchymal cells isolated from lungs of human subjects with IPF (SEE FIG. 2D), a chronic fibrosing and ultimately fatal lung disease. These data indicate a role for TGF-β1 signaling via ALK5/SMAD3 in the induction and activation of NOX4 in myofibroblasts. Myofibroblasts contribute to the tissue repair by secreting ECM proteins and remodeling/contracting the ECM (Tomasek et al. Nat Rev Mol Cell Biol 3, 349-363 (2002), Hinz et al. Am J Pathol 170, 1807-1816 (2007), herein incorporated by reference in their entireties). A 3D-collagen matrix cell culture system was utilized to determine if fibronectin synthesis and contractile functions of myofibroblasts are regulated by NOX4 activation and extracellular H₂O₂ generation. The upregulation of α-smooth muscle actin (α-SMA), a cytoskeletal component of contractile actin stress fibers, and fibronectin synthesis induced by TGF-β1 were inhibited by addition of catalase (SEE FIG. 1G), an enzyme which reduces H₂O₂ to H₂O, indicating a role for H₂O₂ in mediating these effects. Similar to the effects of catalase, endogenous suppression of NOX4 by siRNA knockdown inhibited TGF-β1-induced expression of α-SMA, fibronectin, and procollagen-I (SEE FIG. 1H). Both the exogenous introduction of catalase and RNAi-mediated knockdown of NOX4 inhibited TGF-β1-induced collagen gel contractility (SEE FIGS. 1I and J). Experiments performed during development of embodiments of the present invention indicate a critical role for NOX4-dependent H₂O₂ in conferring synthetic and contractile properties to myofibroblasts that differentiate under the influence of TGF-β1.

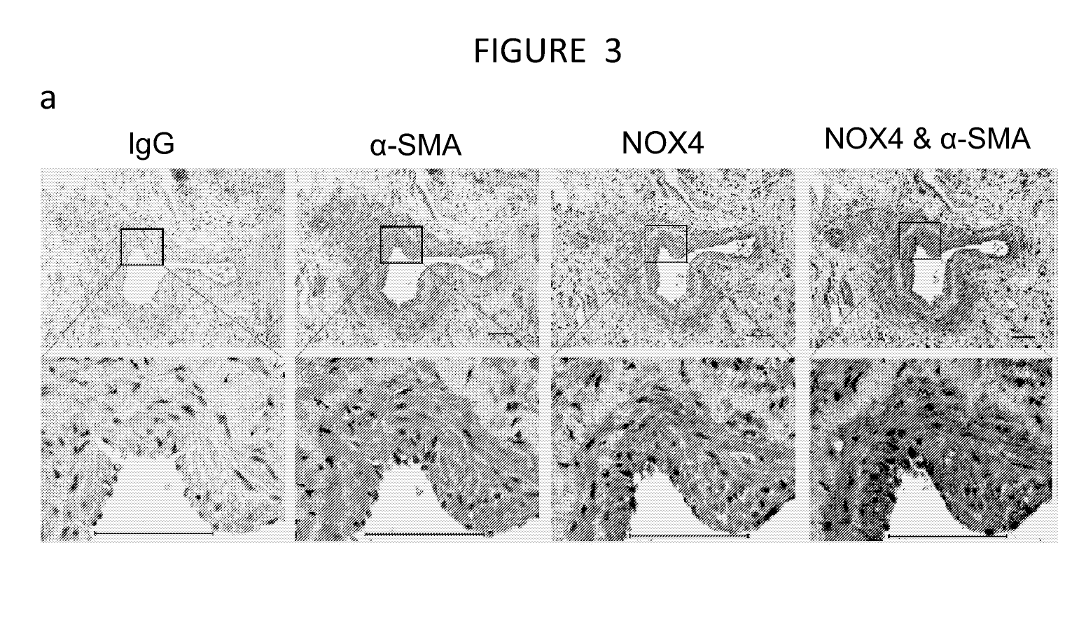

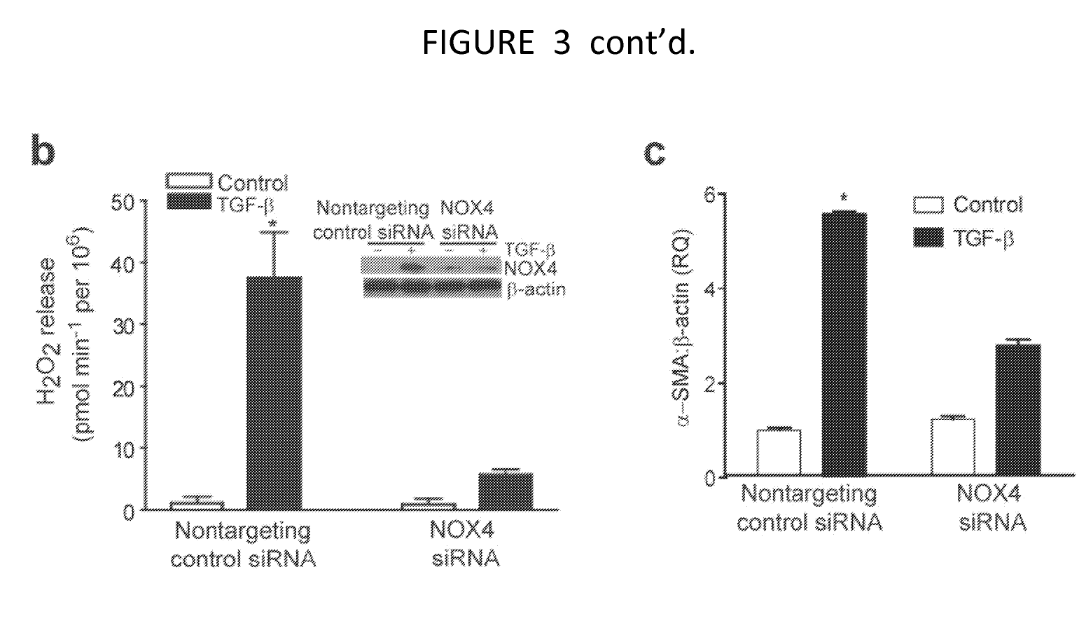

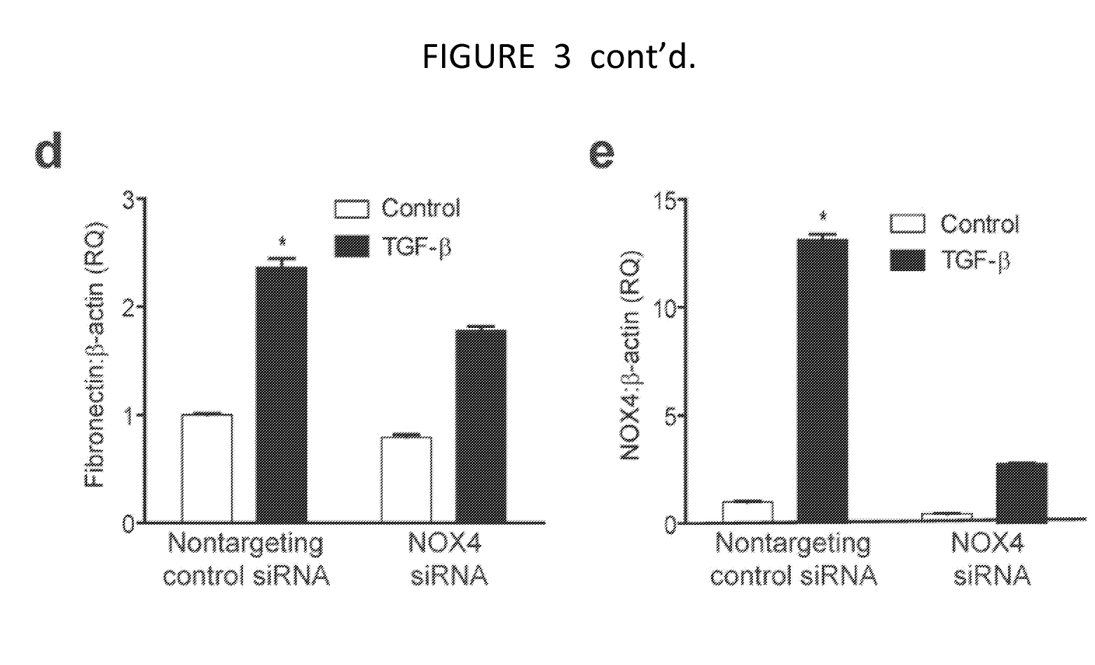

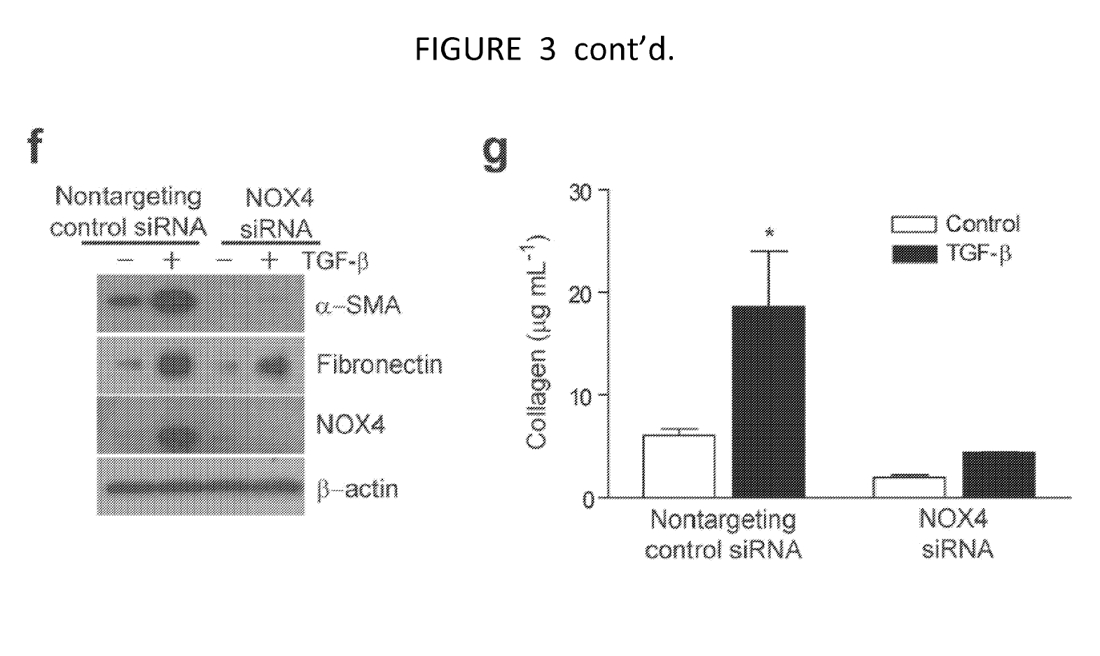

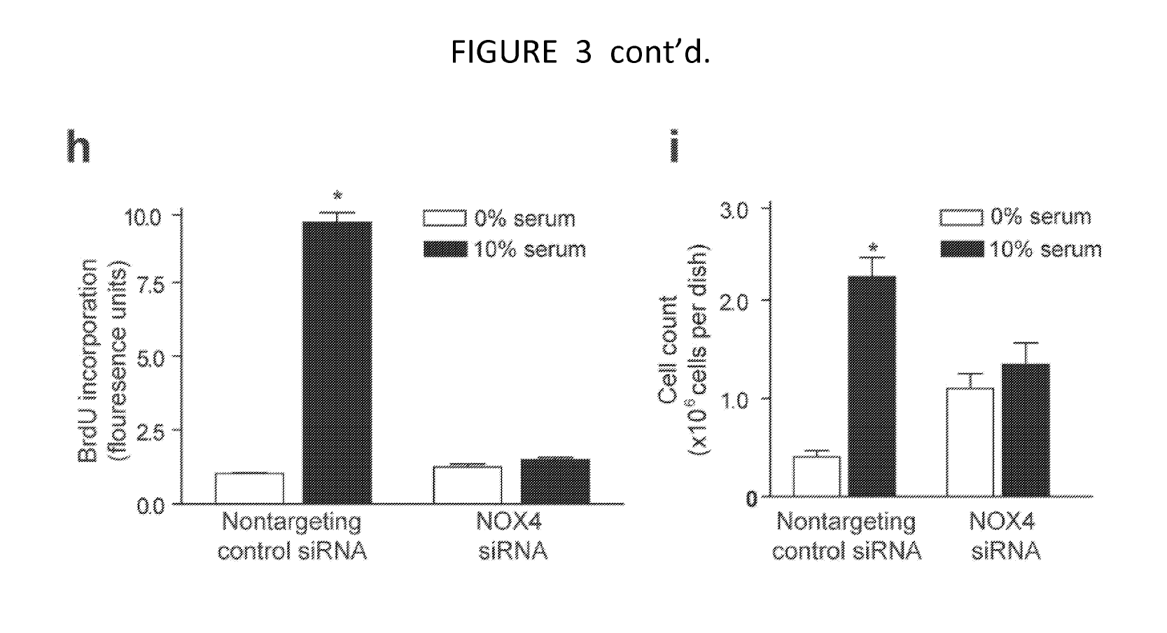

To investigate the role of NOX4 in a human fibrotic disease, lung tissue sections from IPF were examined. NOX4 is highly expressed in myofibroblastic foci of the remodeled IPF lung, as determined by immunohistochemical (IHC) staining (SEE FIG. 3A). Additionally, lung mesenchymal cells isolated from explants of IPF lung tissue (IPF-MCs) were studied ex vivo. Similar to hFLMCs, NOX4 was induced and necessary for TGF-β1-stimulated H₂O₂ production (SEE FIG. 3B); NOX4 was also required for the induction of α-SMA and fibronectin mRNA (SEE FIG. 3C-E) and protein expression (SEE FIG. 3F) by TGF-β1. Differentiated myofibroblasts secrete higher amounts of extracellular collagen. The role of NOX4 on collagen secretion was investigated by IPF-MCs. Constitutive and TGF-β1-induced secretion of soluble collagen by IPF-MCs were inhibited by siRNA-mediated knockdown of NOX4 (SEE FIG. 3G).

The role of NOX4 in IPF-MC proliferation in response to serum was examined; siRNA-mediated knockdown of NOX4 inhibited serum-stimulated proliferation of IPF-MCs (SEE FIGS. 3 H and I). These data indicate NOX4 plays a critical role in myofibroblast differentiation and proliferation of human IPF-MCs.

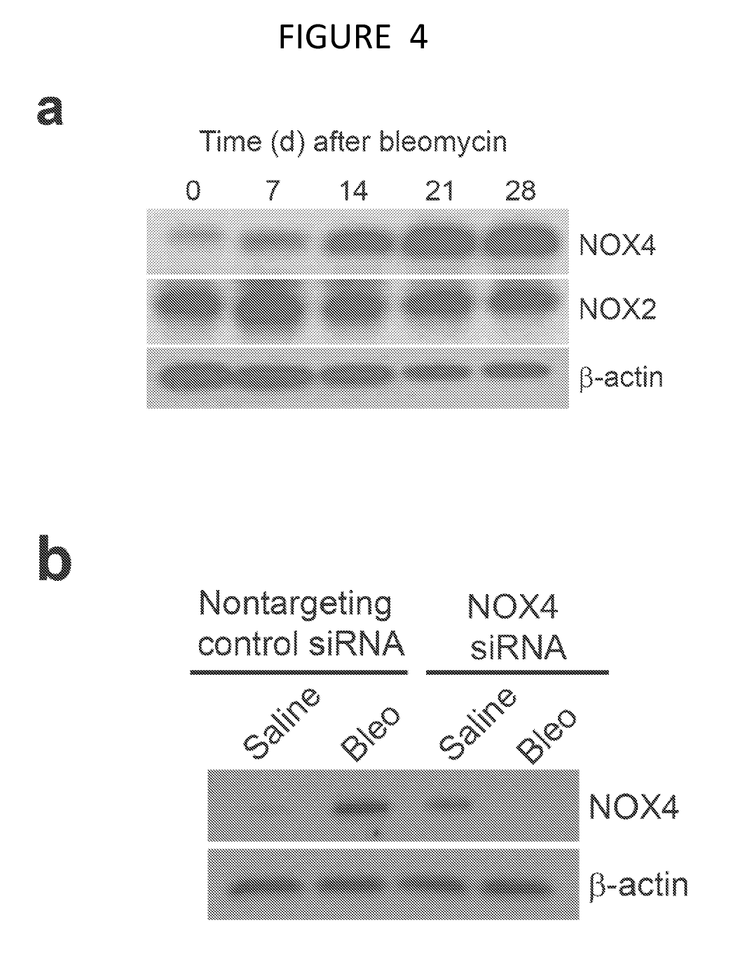

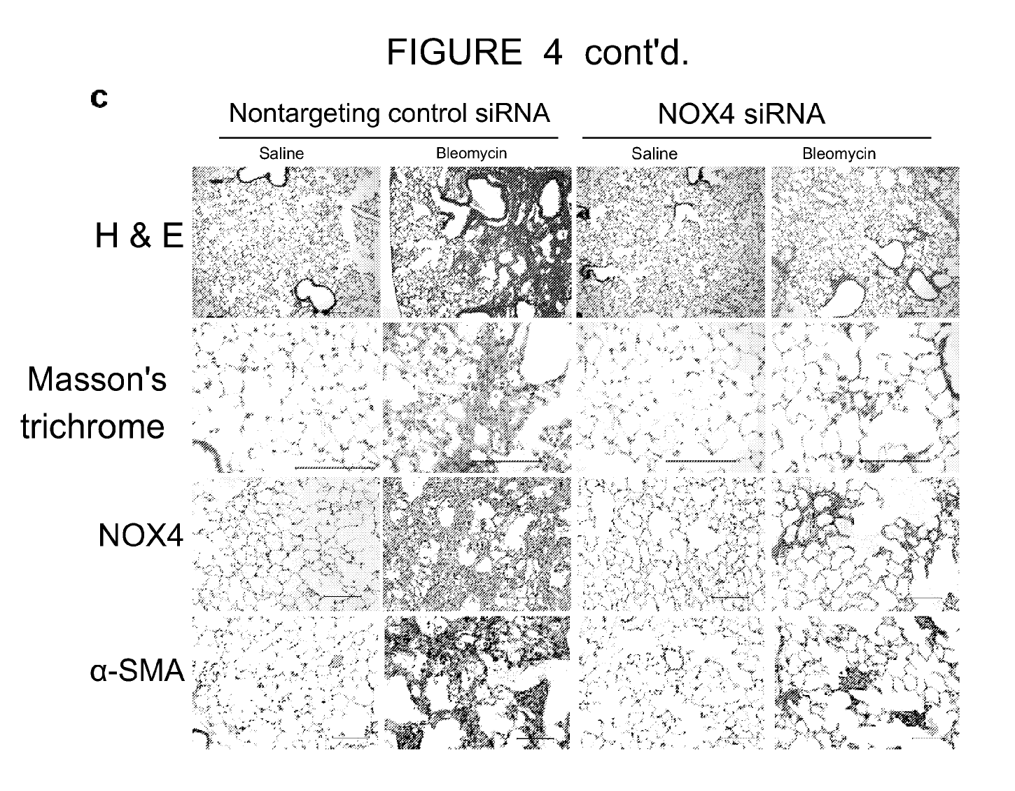

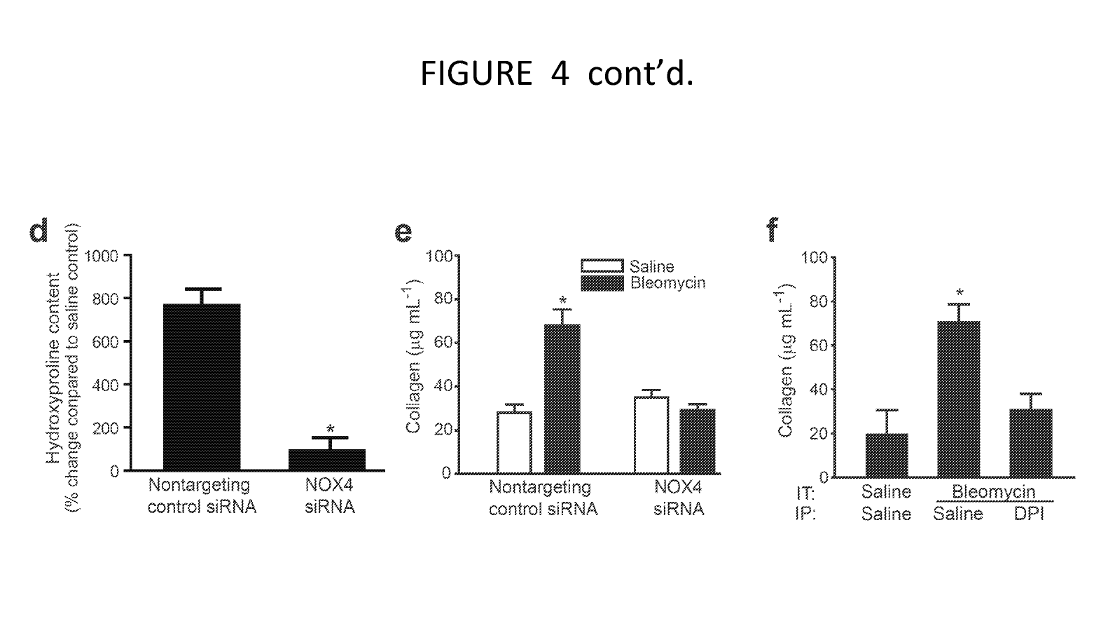

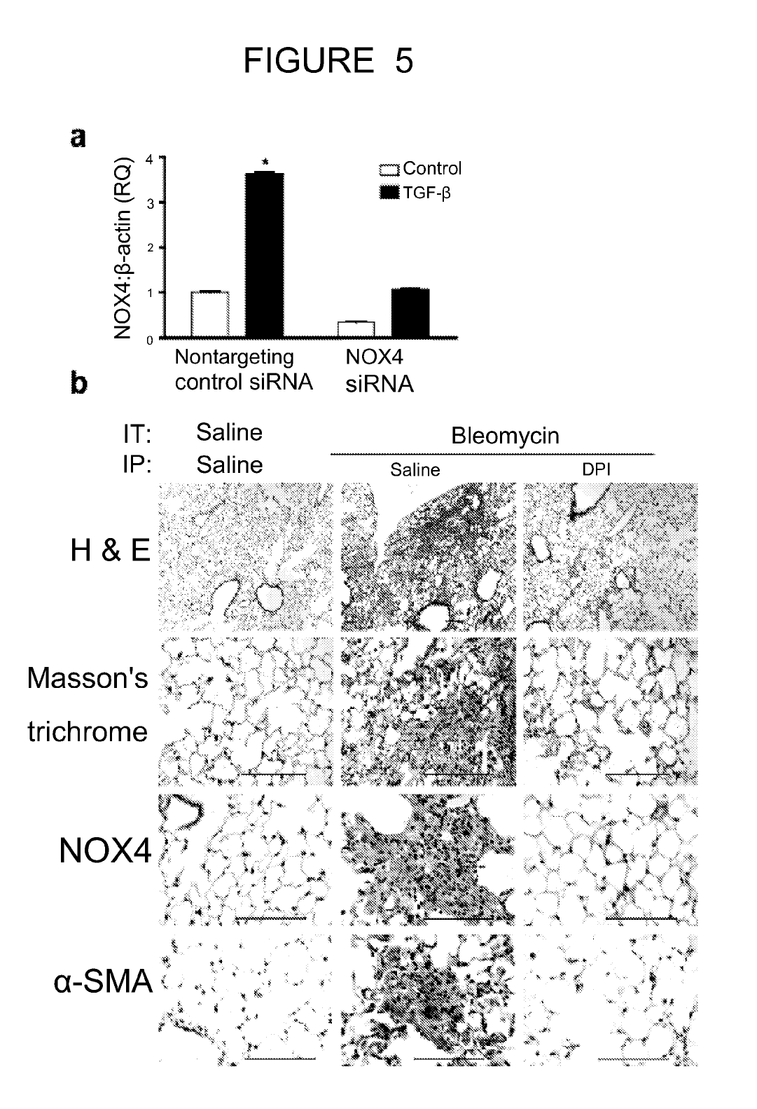

To define the in vivo role of NOX4 in the reparative response to injury of the mammalian lung, a murine model of acute lung injury was employed. In this model, direct airway instillation of the chemotherapeutic drug, bleomycin, causes epithelial injury with subsequent mesenchymal cell activation and fibrosis. Several key features of fibrotic reactions in mammalian tissues, including TGF-β1 upregulation/activation and myofibroblast differentiation/activation are recapitulated in this animal model 20. To determine whether NOX4 expression is induced during the fibrogenic phase of bleomycin-induced lung injury, NOX4 was induced in a time-dependent manner, increasing from day 7 up to day 28 (SEE FIG. 4A), supporting a temporal relationship between NOX4 expression, myofibroblast activation and fibrosis following lung injury. In contrast, expression of the NOX2 isoform, which is predominantly expressed in phagocytic cells, was increased on day 7 and returned to baseline levels at later time-points when inflammatory responses have subsided (SEE FIG. 4A). The effects of targeted suppression of NOX4 induction employing an in vivo RNAi approach in two different animal models of lung injury/fibrosis were examined. The murine siRNA homologous to the human NOX4 siRNA duplex 4 (SEE Table 2) efficiently knocked down NOX4 in cultured primary murine mesenchymal cells (SEE FIG. 5A).

TABLE 2
Human and Murine siRNA and PCR Primers

| | | | |
|---|---|---|---|
| Target Gene/ | | Sense/ | |
| Accession Number | Duplex | Anti-sense | siRNA Sequence |

(a) siRNA Sequences - Human

| | | | |
|---|---|---|---|
| Nox4 | 1 | Sense | GAAUUACAGUGAAGACUUU |
| NM_016931 | | Anti-sense | AAAGUCUUCACUGUAAUUC |
| Nox4 | 2 | Sense | CAGGAGGGCUGCUGAAGUA |
| NM_016931 | | Anti-sense | UACUUCAGCAGCCCUCCUG |
| Nox4 | 3 | Sense | GGGCUAGGAUUGUGUCUAA |
| NM_016931 | | Anti-sense | UUAGACACAAUCCUAGCCC |
| Nox4 | 4 | Sense | GAUCACAGCCUCUACAUAU |
| NM_016931 | | Anti-sense | AUAUGUAGAGGCUGUGAUC |
| SMAD3 | N/A | Sense | UAGGCAGAAGCGCUCCGAA |
| NM_005902 | | Anti-sense | UUCGGAGCGCUUCUGCCUA |

| | | |
|---|---|---|
| Target Gene / | Sense/ | |
| Accession Number | Anti-sense | siRNA Sequence |

(b) siRNA Sequences - Mouse

| Nox4 | Sense | | GGUUACAGCUUCUACCUAC |
|---|---|---|---|
| NM_015760 | Anti-sense | | GUAGGUAGAAGCUGUAACC |
| Target Gene/ | Forward/ | | Product |
| Accession Number | Reverse | Primer Sequence | Size |

(c) RT-PCR Primers - Human

| β-actin | Forward | CACCCTGAAGTACCCCATCGA | 448 |
|---|---|---|---|
| NM_001101 | Reverse | CTCCTTAATGTCACGCACGATTTC | |
| Nox4 | Forward | CTGGAGGAGCTGGCTCGCCAACGAAG | 516 |
| NM_016931 | Reverse | GTGATCATGAGGAATAGCACCACCACCATGC | |
| | | AG | |
| Target Gene/ | Forward/ | | Product |
| Accession Number | Reverse | Primer Sequence | Size |

(d) Real-Time PCR Primers - Human

| β-actin | Forward | TGCTATCCAGGCTGTGCTAT | 62 |
|---|---|---|---|
| NM_001101 | Reverse | AGTCCATCACGATGCCAGT | |
| Nox4 | Forward | AGATGTTGGGGCTAGGATTG | 137 |
| NM_016931 | Reverse | TCTCCTGCTTGGAACCTTCT | |
| α-SMA | Forward | GACCGAATGCAGAAGGAGAT | 98 |
| NM_001613 | Reverse | CCACCGATCCAGACAGAGTA | |
| Fibronectin 1 | Forward | GTGGCTGAAGACACAAGGAA | 145 |
| NM_212482 | Reverse | CCTGCCATTGTAGGTGAAT | |
| Target Gene/ | Forward/ | | Product |
| Accession Number | Reverse | Primer Sequence | Size |

(e) Real-Time PCR Primers - Mouse

| | | | |
|---|---|---|---|
| β-actin | Forward | AGTGTGACGTTGACATCCGT | 120 |
| NM_007393 | Reverse | TGCTAGGAGCCAGAGCAGTA | |
| Nox4 | Forward | ACTTTTCATTGGGCGTCCTC | 100 |
| NM_015760 | Reverse | AGAACTGGGTCCACAGCAGA | |
| α-SMA | Forward | GTCCCAGACATCAGGGAGTAA | 102 |
| NM_007392 | Reverse | TCGGATACTTCAGCGTCAGGA | |
| Fibronectin 1 | Forward | ACCGACAGTGGTGTGGTCTA | 130 |
| NM_010233 | Reverse | CACCATAAGTCTGGGTCACG | |

In the first animal model, NOX4 siRNA or nontargeting control siRNA was instilled directly down the trachea of mice at the time of bleomycin injury (day 0), and tissues were analyzed at day 14 or 21. NOX4 siRNA was effective in inhibiting NOX4 induction in injured lung tissue at 21 days as determined by Western immunoblotting (SEE FIG. 4B), and at day 14 as determined by IHC analysis (SEE FIG. 4C). IHC analysis confirmed NOX4 expression localized to fibrotic foci surrounding remodeled alveolar structures on day 14 post-lung injury (SEE FIG. 4C). NOX4 knockdown mediated a marked anti-fibrotic effect as determined by histopathology, trichrome staining for collagen, IHC analysis of α-SMA (SEE FIG. 4C), and by biochemical analyses of hydroxyproline content (SEE FIG. 4D) and acid-soluble collagen (SEE FIG. 4E) in whole lung homogenates. Pharmacological approaches that specifically target fibrogenic processes were lacking in the field prior to the present invention. The flavoenzyme inhibitor, diphenyleneiodonium chloride (DPI), was employed to determine if pharmacologic blockade of NOX/flavoenzyme activity during the postinjury reparative phase protects against fibrogenic tissue responses. DPI, which blocks NOX4 activity in myofibroblasts at a relatively low IC50 (<0.5 μM), was administered by daily intra-peritoneal injection on days 8 to 21 following bleomycin lung injury. DPI administration was delayed until day 8 to minimize effects of the drug on inflammatory responses that typically subside following the first week bleomycin lung injury in mice (Thrall et al. Am Rev Respir Dis 126, 488-492 (1982), Vittal et al. Am J Pathol 166, 367-375 (2005), herein incorporated by reference in their entirety). Mice receiving DPI demonstrated significant protection from fibrosis, as measured by acid-soluble collagen in whole lung (SEE FIG. 4F); this is associated with reduced fibrosis as determined by histopathology, Masson's trichrome staining for collagen, as well as reduced expression of NOX4 and α-SMA expressing myofibroblasts (SEE FIG. 5B).

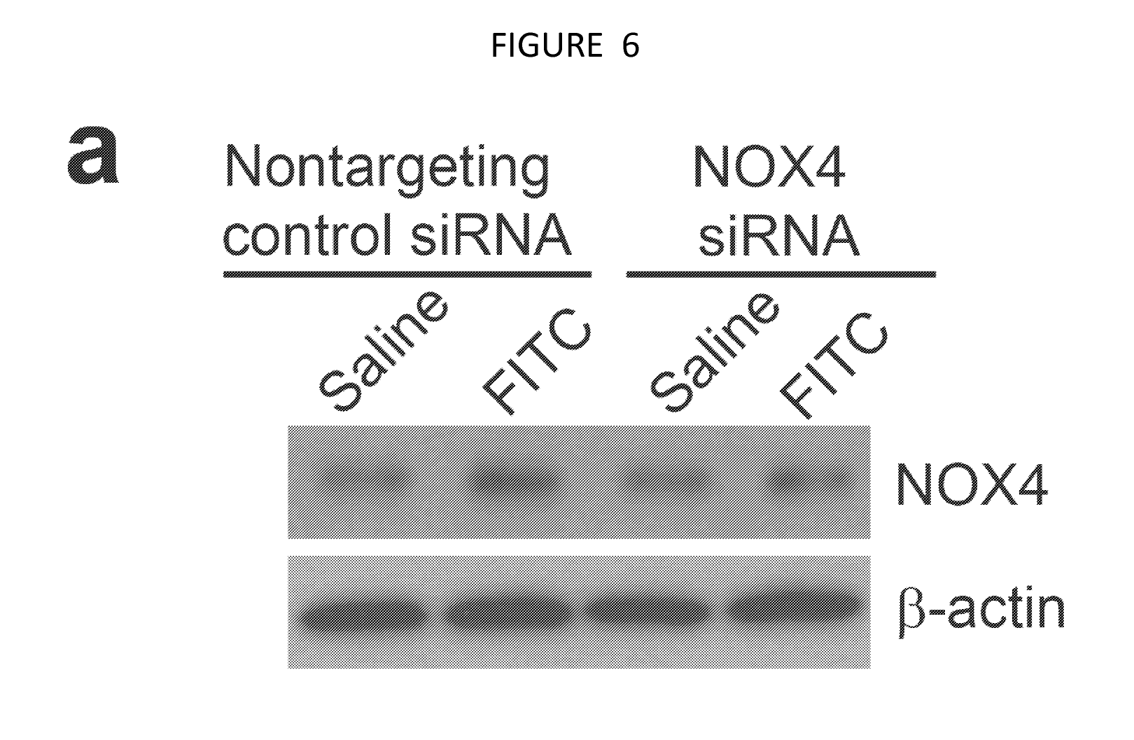

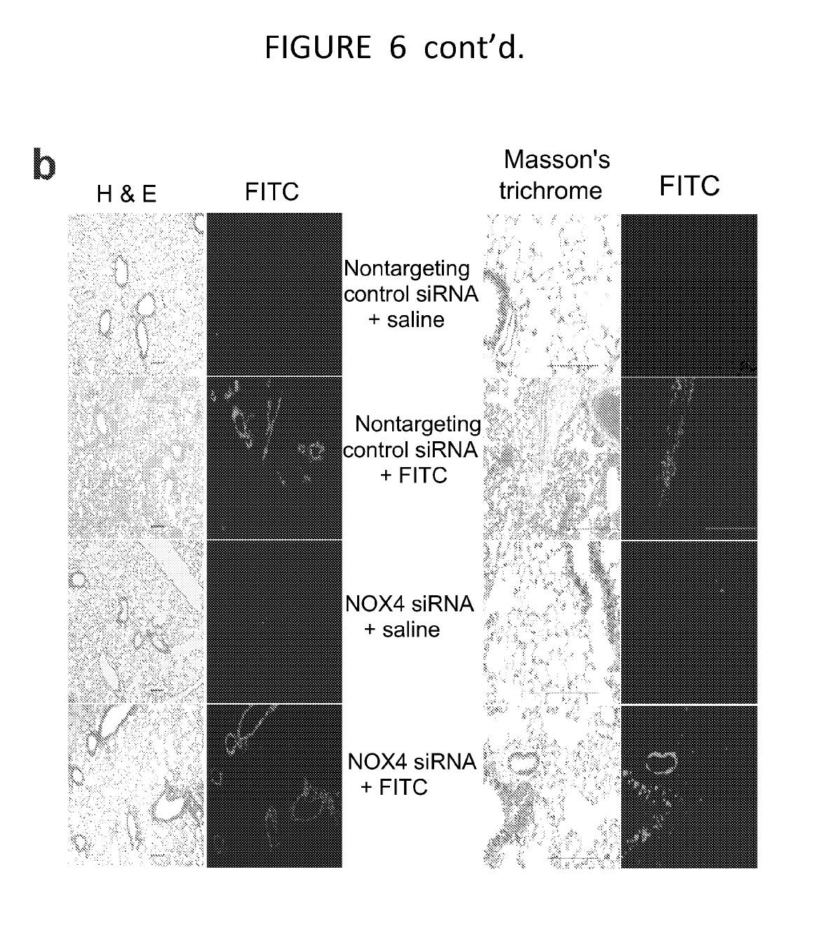

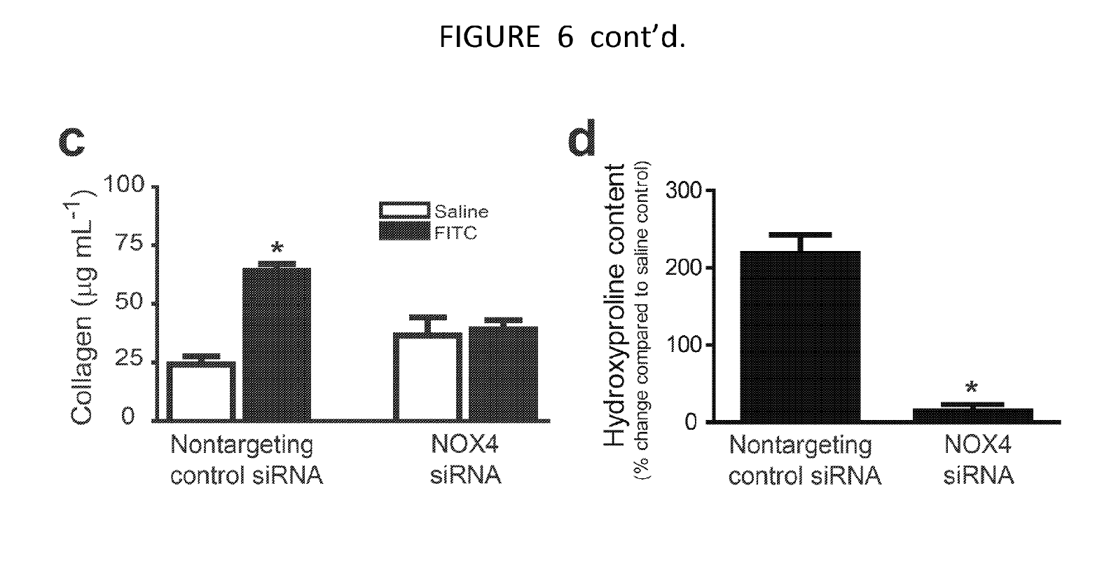

A hapten (fluorescein isothiocyanate; FITC)-driven lung injury/fibrosis murine model was employed to test the role of NOX4 in fibrogenesis (Roberts et al. J Pathol 176, 309-318 (1995), herein incorporated by reference in its entirety). In this model, NOX4 siRNA was instilled directly down the trachea of mice at the time of FITC injury (day 0), and tissues were analyzed on day 14 or 21. This RNAi approach was effective in inhibiting NOX4 induction at day 21 post-lung injury (SEE FIG. 6A). The fibrotic response surrounding airways where FITC is deposited was found to be markedly attenuated in mice receiving NOX4 siRNA versus nontargeting siRNA, as determined by histopathology and trichrome staining for collagen (SEE FIG. 6B); this was confirmed by analyzing whole lung homogenates for acid-soluble collagen by Sircol assay (SEE FIG. 6C) and acid-insoluble collagen by hydroxyproline assay (SEE FIG. 6D).

In experiments performed during development of embodiments of the present invention, a functional role was demonstrated for NOX4 in myofibroblast differentiation and activation ex vivo and in fibrogenic responses to lung injury in vivo. The pro-fibrogenic mediator, TGF-β1, specifically induces mRNA/protein expression and enzymatic activation of the NOX4 isoform in differentiated myofibroblasts. NOX4-dependent H₂O₂ generation is required for myofibroblast differentiation, synthesis of ECM proteins, and contractility mediated by TGF-β1. NOX4 is expressed in myofibroblastic foci of remodeled IPF lung tissue, supporting a role for this NOX isoform in the induction and activation of myofibroblasts in this human pulmonary disorder. In two different murine models of pulmonary fibrosis, genetic or pharmacologic strategies targeting NOX4 induction or activity protects against fibrosis. An RNAi strategy was utilized to suppress NOX4 expression by administering NOX4 siRNA at the time of bleomycin or FITC injury. Although the present invention is not limited to any particular mechanism of action and an understanding of the mechanism of action is not necessary to practice the present invention, the disruption of the airway-alveolar epithelial barrier immediately following bleomycin/FITC injury facilitated the transduction of siRNA to epithelial or mesenchymal precursor cells that prevented their differentiation into activated myofibroblasts, thus protecting from pulmonary fibrosis. Experiments conducted during development of embodiments of the present invention are the first to definitively implicate a specific NOX isoform in tissue repair functions and fibrogenesis. NOX4 may have been selected during metazoan evolution to execute tissue repair functions critical for the survival of more complex multicellular eukaryotes (Thannickal. Am J Respir Cell Mol Biol 40, 507-510 (2009), herein incorporated by reference in its entirety). In support of this notion, NOX4 is almost exclusively expressed in chordates (Sumimoto. Febs J 275, 3249-3277 (2008), herein incorporated by reference in its entirety). Furthermore, NOX4 activation in myofibroblasts and tissue fibrogenesis may represent yet another example of antagonistic pleiotropy, whereby genes that confer a survival advantage during early reproductive life mediate potential harmful effects in later life (Lambeth. Free Radic Biol Med 43, 332-347 (2007), herein incorporated by reference in its entirety). Fibrosis is typically a complication of failed tissue regeneration and ineffective epithelial repair in diverse organ systems, often with an age-dependent increase in incidence (Raghu et al. Am J Respir Crit. Care Med 174, 810-816 (2006), herein incorporated by reference in its entirety).

Fibrosis in mammalian tissues is perhaps best viewed as an initial adaptive response executed by mesenchymal cells to restore tissue barrier function while secreting a provisional matrix to facilitate re-epithelialization; however, persistent mesenchymal activation and failed reepithelialization results in unrestrained and progressive fibrosis. Targeting NOX4 provides a therapeutic strategy for an otherwise treatment-unresponsive and ultimately fatal group of human fibrotic disorders.

We claim:

1. A method of treating or preventing pulmonary fibrosis in a subject, comprising: inhibiting NOX4 in said subject.

2. The method of claim 1, wherein said subject is at risk for pulmonary fibrosis.

3. The method of claim 1, wherein said subject suffers from pulmonary fibrosis.

4. The method of claim 1, wherein inhibiting NOX4 comprises inhibiting expression of the NOX4 gene.

5. The method of claim 1, wherein inhibiting NOX4 comprises inhibiting NOX4 enzymatic activity.

6. The method of claim 1, wherein inhibiting NOX4 comprises administering one or more NOX4-inhibiting agents to said subject.

7. The method of claim 6, wherein inhibiting NOX4 comprises administering siRNA to said subject.

8. The method of claim 7, wherein said siRNA comprise one or more of SEQ ID NOs:1-10.

9. The method of claim 6, wherein inhibiting NOX4 comprises administering one or more small molecule drugs to said subject.

10. The method of claim 6, wherein inhibiting NOX4 comprises administering antibodies to said subject.

11. The method of claim 1, wherein said pulmonary fibrosis is unresponsive to one or more other standard treatments.

12. The method of claim 1, further comprising administering a second anti-fibrotic agent to said subject.
